(19) United States
(12) Patent Application Publication
(10) Pub. No.: US 2022/0348985 A1
(43) Pub. Date: Nov. 3, 2022

(54) FORCED INTERCALATION (FIT)-APTAMERS: PROBES BASED ON FORCED INTERCALATION

(71) Applicant: NORTHWESTERN UNIVERSITY, Evanston, IL (US)

(72) Inventors: Chad A. Mirkin, Wilmette, IL (US); Sasha B. Ebrahimi, Dekalb, IL (US); Devleena Samanta, Evanston, IL (US); Ho Fung Cheng, Evanston, IL (US)

(21) Appl. No.: 17/621,088

(22) PCT Filed: Jun. 19, 2020
(86) PCT No.: PCT/US20/38778

§ 371 Date: Dec. 20, 2021

Related U.S. Application Data
(60) Provisional application No. 62/864,854, filed on Jun. 21, 2019.

(51) Int. Cl. C12Q 1/6804; C12Q 1/6816

(52) U.S. Cl. CPC C12Q 1/6804; C12Q 1/6816

(57) ABSTRACT

The present disclosure is directed to aptamers comprising a detectable marker situated at an internal location within the aptamer, use of the aptamers to, e.g., detect target analytes, and methods of making the aptamers. In exemplary embodiments, methods of the disclosure comprise contacting the target analyte with an aptamer comprising a detectable marker situated at an internal location within the aptamer, wherein the contacting results in binding of the target analyte to the aptamer, wherein target analyte binding to the aptamer results in restriction of internal rotation of the marker, resulting in a detectable change in the marker.

### CROSS-REFERENCE TO RELATED APPLICATIONS

This application claims the priority benefit under 35 U.S.C. § 119(e) of U.S. Provisional Patent Application No. 62/864,854, filed Jun. 21, 2019, which in incorporated herein by reference in its entirety.

### STATEMENT OF GOVERNMENT INTEREST

This invention was made with government support under FA8650-15-2-5518 awarded by the Air Force Research Laboratory (AFRL), N00014-15-1-0043 awarded by the Office of Naval Research (ONR), and U54 CA199091 awarded by the National Institutes of Health (NIH). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2019-117_Seqlisting.txt”, which was created on Jun. 19, 2020 and is 8,676 bytes in size. The subject matter of the Sequence Listing is incorporated herein in its entirety by reference.

### BACKGROUND

Aptamers, oligonucleotide sequences that can be evolved to bind to analytes with high sensitivity and specificity, have recently found widespread use as effective therapeutic and diagnostic tools.¹⁻⁴ To be used as a tool for detection, the binding of an aptamer to its target must result in a signaling event that can be monitored as a readout for target presence. Fluorescence-based techniques have emerged as popular readout platforms due to their simplicity, low-cost, high-throughput, and ability to multiplex.⁵⁻¹⁵ For example, several strategies have been designed wherein target binding to an aptamer labeled with a fluorophore-quencher pair induces a structural change that separates the fluorophore and the quencher (e.g. structure-switching signaling aptamers,⁶ aptamer beacons,⁸ aptamer switch probes⁹). Alternatively, constructs that bring a pair of dyes into close proximity upon target binding to elicit a fluorescence signal by Forster resonance energy transfer (FRET) are also commonly employed.¹⁰ More recently, Spinach aptamers and variants thereof have been developed that change structure after aptamer-target complexation, allowing a small molecule fluorophore to bind to the Spinach region in the sensing unit and yield fluorescence turn-on.¹¹⁻¹⁴

While these methods constitute a powerful means to detect targets of interest, they also suffer from limitations. Strategies that rely on partial blocking of the aptamer site (i.e. structure-switching aptamers, aptamer beacons) have retarded aptamer-target binding kinetics, increasing the time required to get a readout.¹⁶ Systems based on fluorophore/quencher pairs are prone to false-positive signals in complex media and cells due in part to nuclease degradation.¹⁷ Moreover, strategies based on FRET are generally associated with low signal to noise ratios.¹⁸ Platforms like Spinach require long sequences to be appended to aptamers, making their folding and, therefore, efficacy difficult to predict in complex milieu.¹⁹

### SUMMARY

Detecting and studying analytes in complex media and living cells is an outstanding challenge. The present disclosure is directed to aptamers, oligonucleotide sequences which can bind to analytes of interest, to study various targets. The disclosure offers a new signal transduction method for target presence readout. The platform described herein can be extended to the detection of any target analyte (including but not limited to ions, small molecules, lipids, carbohydrates, oligosaccharides, and proteins), offering a simple and powerful approach to study analytes of interest. Provided herein is a fundamentally new design strategy for interfacing aptamers with a readout event via viscosity-sensitive fluorophores. It has been shown that dyes of the thiazole orange family can be covalently attached to mRNA recognition sequences to create “duplex-sensitive” fluorescence turn-on probes.²⁰⁻²³ The fluorescence enhancement stems from the restricted rotation of the dye around its methine bridge upon forced intercalation (FIT) in the oligonucleotide duplex. Notably, these probes avoid false-positive signals because their turn-on does not rely on proximity between a fluorophore and a quencher. It is disclosed herein, however, that by strategically placing the dye in an aptamer sequence such that structural changes of the aptamer upon ligand binding hinders the dye's internal rotation, a new class of false-positive resistant signaling aptamers can be designed (FIG. 1). Additionally, these “FIT-aptamers” respond faster compared to probes relying on partial blocking of the aptamer site and require only a single modification unlike Spinach-based platforms.¹²

In particular, a general design strategy is disclosed that transduces an aptamer-target binding event into a fluorescence readout via the use of a viscosity-sensitive dye. Target binding to the aptamer leads to restriction of the internal rotation of the dye (e.g., through forced intercalation (FIT) of the dye between oligonucleotide base pairs), increasing its fluorescence. In some embodiments, the fluorescence is increased by up to 20-fold.

Specifically, it is demonstrated herein that FIT-aptamers can report target presence through intramolecular conformational changes, sandwich assays, as well as target-templated reassociation of split-aptamers, showing that the most common aptamer-target binding modes can be coupled to a FIT-based readout. This strategy also can be used to detect the formation of a metallo-base pair within a duplexed strand and is therefore attractive for screening for metal-mediated base pairing events. Importantly, FIT-aptamers reduce false-positive signals typically associated with fluorophore-quencher based systems, quantitatively outperform FRET-based probes, and allow rapid and highly sensitive target detection in complex media such as human serum. Taken together, FIT-aptamers are a new class of signaling aptamers which contain a single modification, yet detect a broad range of targets.

Applications of the technology disclosed herein include, but are not limited to, detecting and imaging analytes in cells and complex media, quantifying levels of analytes in cells and complex media, imaging analytes with spatiotemporal resolution in living cells, regulating and detecting analytes of interest (theranostic), screening for new metallo base pairs, and studying processes that lead to local conformational changes in nucleic acid sequences.

Strategies that rely on partial blocking of the aptamer site have reduced aptamer-target binding kinetics, increasing the time required to get a readout. FIT-aptamers, however, do not require a design that partially blocks the aptamer binding site, leading to superior readout kinetics. Further, systems based on fluorophore/quencher pairs are prone to false-positive signals in complex media and cells due in part to nuclease degradation. FIT-aptamers do not require a quencher and are therefore resistant to false-positive signal.

Strategies based on FRET are generally associated with low signal to noise ratios, but FIT-aptamers do not rely on FRET for signal readout and have superior signal to noise ratios in comparison to FRET.

Platforms like Spinach require long sequences to be appended to aptamers, making their folding and, therefore, efficacy difficult to predict in complex milieu. FIT-aptamers only require a single modification, leading to simpler probes.

Accordingly, FIT-aptamers are the only detectable aptamer probe that can detect almost any target of interest with a single modification.

The present disclosure provides a fundamentally new design strategy for interfacing aptamers with a readout event via viscosity-sensitive fluorophores. The fluorescence of these fluorophores is enhanced when rotation about the methine bridge is restricted. The signaling aptamers of the disclosure were designed by strategically placing the dye in an aptamer sequence such that structural changes of the aptamer upon ligand binding hinder the dye's internal rotation leading to forced intercalation (FIT).

In some aspects, the disclosure provides a method of detecting the presence of a target analyte comprising the step of contacting the target analyte with an aptamer comprising a detectable marker situated at an internal location within the aptamer, wherein the contacting results in binding of the target analyte to the aptamer, wherein target analyte binding to the aptamer results in restriction of internal rotation of the marker, resulting in a detectable change in the marker. In some embodiments, target analyte binding to the aptamer results in forced intercalation (FIT) of the marker between oligonucleotide base pairs of the aptamer. In some aspects, the disclosure provides a method of detecting the presence of a target analyte comprising the step of contacting the target analyte with (a) an aptamer or portion thereof comprising (i) nucleotide sequence X, (ii) nucleotide sequence Y which binds to the target analyte, either alone or in combination with nucleotide sequence Y′ and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change in the marker. In some embodiments, nucleotide sequence Y and nucleotide sequence Y′ bind to different binding sites of the target analyte. In some embodiments, nucleotide sequence Y and nucleotide sequence Y′ together bind to the same binding site of the target analyte. In some embodiments, the binding of nucleotide sequence X with nucleotide sequence X′ and the target analyte with nucleotide sequence Y and nucleotide sequence Y′ result in forced intercalation (FIT) of the marker between oligonucleotide base pairs of the aptamer and the additional aptamer. In various embodiments, the detectable marker is a marker with internal rotation-dependent fluorescence. In some embodiments, the detectable marker is a viscosity-sensitive marker. In various embodiments, the detectable marker is thiazole orange (TO), quinoline blue, quinoline violet, thiazole red, a derivative thereof, or a cyanine derivative. In further embodiments, the change in the detectable marker is proportional to concentration of the target analyte. In some embodiments, the target analyte is a protein, an ion, a small molecule, a lipid, a carbohydrate, an oligosaccharide, a cell, or a combination thereof. In some embodiments, the ion is a metal ion. In further embodiments, the metal ion is a mercury ion, a copper ion, a silver ion, zinc ion, gold ion, manganese ion, or a combination thereof. In some embodiments, the ion is a hydrogen ion. In some embodiments, the change in the detectable marker is indicative of a pH change. In some embodiments, the aptamer is a DNA aptamer, an RNA aptamer, or a modified form thereof. In some embodiments, the additional aptamer is a DNA aptamer, an RNA aptamer, or a modified form thereof. In some embodiments, the aptamer is about 5 to about 1000 nucleotides in length. In some embodiments, the aptamer is about 10 to about 100 nucleotides in length. In some embodiments, the additional aptamer is about 5 to about 1000 nucleotides in length. In some embodiments, the additional aptamer is about 10 to about 100 nucleotides in length. In some embodiments, the aptamer comprises a spacer. In some embodiments, the additional aptamer comprises a spacer. In some embodiments, the detectable marker is situated at a position that is x nucleotides from a terminus of the aptamer, wherein x is an integer that is 1, n/2, or any integer between 1 and n/2, wherein n is (i) the length of the aptamer and (ii) an even number. In some embodiments, the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number. In some embodiments, the detectable marker is situated at a position that is 1, 2, 3, 4, 5, 6, 7, 8, 9, 10, or more nucleotides from a terminus of the aptamer.

In some aspects, the disclosure provides a method of identifying a non-canonical base pair comprising the step of contacting an ion with (a) a first oligonucleotide comprising a detectable marker situated at an internal location within the first oligonucleotide and (b) a second oligonucleotide, wherein the first oligonucleotide and the second oligonucleotide are sufficiently complementary to hybridize to each other and form a duplex, but are not complementary at a position in the duplex immediately adjacent to the detectable marker when the first oligonucleotide is hybridized to the second oligonucleotide; and wherein binding of the ion to the duplex results in restriction in the internal rotation of the marker, resulting in a detectable change in the marker and thereby identifying the non-canonical base pair. In some embodiments, binding of the ion to the duplex results in forced intercalation (FIT) of the marker in the duplex. In some embodiments, the detectable marker is a marker with internal rotation-dependent fluorescence. In some embodiments, the detectable marker with internal rotation-dependent fluorescence is a viscosity-sensitive marker. In some embodiments, the detectable marker is thiazole orange (TO), quinoline blue, quinoline violet, thiazole red, a derivative thereof, a cyanine derivative, or a combination thereof. In some embodiments, the ion is a cation. In some embodiments, the cation is a metal ion. In some embodiments, the metal ion is a mercury ion, a copper ion, a silver ion, zinc ion, gold ion, manganese ion, or a combination thereof. In some embodiments, the ion is an anion. In some embodiments, the first oligonucleotide is DNA, RNA, or a modified form thereof. In some embodiments, the second oligonucleotide is DNA, RNA, or a modified form thereof. In some embodiments, the first oligonucleotide is about 5 to about 1000 nucleotides in length. In some embodiments, the first oligonucleotide is about 10 to about 100 nucleotides in length. In some embodiments, the second oligonucleotide is about 5 to about 1000 nucleotides in length. In some embodiments, the second oligonucleotide is about 10 to about 100 nucleotides in length. In some embodiments, the first oligonucleotide comprises a spacer. In some embodiments, the second oligonucleotide comprises a spacer. In some embodiments, the detectable marker is situated at a position that is x nucleotides from a terminus of the first oligonucleotide, wherein x is an integer that is 1, n/2, or any integer between 1 and n/2, wherein n is (i) the length of the first oligonucleotide and (ii) an even number. In some embodiments, the detectable marker is situated at a position that is x nucleotides from a terminus of the first oligonucleotide, wherein x is an integer that is 1, (n+1)/2, or any integer between 1 and (n+1)/2, wherein n is (i) the length of the first oligonucleotide and (ii) an odd number. In some embodiments, the detectable marker is situated at a position that is 1, 2, 3, 4, 5, 6, 7, 8, 9, 10, or more nucleotides from a terminus of the first oligonucleotide. In some embodiments, the first oligonucleotide and the second oligonucleotide are sufficiently complementary to hybridize to each other and form a duplex, but are not complementary at a single position in the duplex immediately adjacent to the detectable marker.

In some aspects, the disclosure provides a method of identifying a non-canonical base pair comprising the step of contacting an ion with an aptamer comprising a detectable marker situated at an internal location within the aptamer, wherein the aptamer is able to form an intramolecular duplex, and the duplex comprises a nucleotide mismatch at a position immediately adjacent to the detectable marker; and wherein binding of the ion to the duplex results in restriction in the internal rotation of the marker, resulting in a detectable change in the marker and thereby identifying the non-canonical base pair. In some embodiments, binding of the ion to the duplex results in forced intercalation (FIT) of the marker in the duplex. In some embodiments, the detectable marker is a marker with internal rotation-dependent fluorescence. In some embodiments, the detectable marker with internal rotation-dependent fluorescence is a viscosity-sensitive marker. In some embodiments, the detectable marker is thiazole orange (TO), quinoline blue, quinoline violet, thiazole red, a derivative thereof, or a cyanine derivative. In some embodiments, the ion is a cation. In some embodiments, the cation is a metal ion. In some embodiments, the metal ion is a mercury ion, a copper ion, a silver ion, zinc ion, gold ion, manganese ion, or a combination thereof. In some embodiments, the ion is an anion. In some embodiments, the aptamer is a DNA aptamer, an RNA aptamer, or a modified form thereof. In some embodiments, the aptamer is about 5 to about 1000 nucleotides in length. In some embodiments, the aptamer is about 10 to about 100 nucleotides in length. In some embodiments, the aptamer comprises a spacer. In some embodiments, the detectable marker is situated at a position that is x nucleotides from a terminus of the aptamer, wherein x is an integer that is 1, n/2, or any integer between 1 and n/2, wherein n is (i) the length of the aptamer and (ii) an even number. In some embodiments, the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number. In some embodiments, the detectable marker is situated at a position that is 1, 2, 3, 4, 5, 6, 7, 8, 9, 10, or more nucleotides from a terminus of the aptamer. In some embodiments, the duplex consists of a nucleotide mismatch at a position immediately adjacent to the detectable marker.

### BRIEF DESCRIPTION OF THE DRAWINGS

FIG. 1 depicts FIT-aptamers. Aptamers modified with a visco-sensitive dye (quinoline blue) fluoresce upon target binding due to target-induced conformational changes.

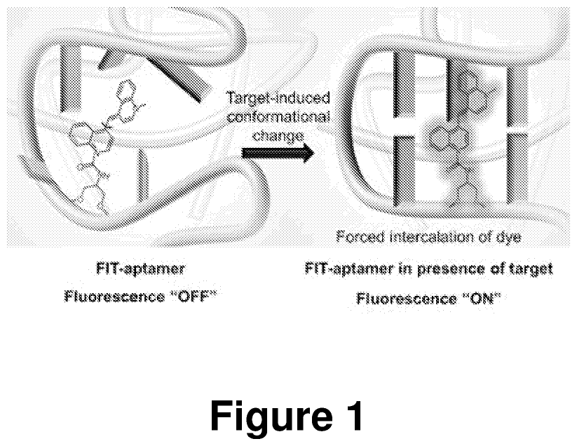

FIG. 2 depicts a scheme of synthesizing an amino-modified oligonucleotide and coupling the FIT-dye to it.

FIG. 3 shows the FIT-aptamer for Hg²⁺. (A) Design scheme using HgA1. (B) Fluorescence enhancement vs [Hg²⁺]. Red line denotes fit to a Hill-equation. (C) Scheme when a short complementary strand (HgA1comp) is used to partially block the Hg²⁺-binding sites on HgA1. (D) Time taken for the fluorescence to change after Hg²⁺ addition to HgA1 in the absence and presence of HgA1comp.

FIG. 4 shows the FIT-aptamer for Hg²⁺ (HgA2). (A) Design scheme. (B) Fluorescence enhancement (I_{f}/I₀) vs [Hg²⁺] added. Black spheres denote experimentally observed values. Red dashed line indicates theoretical fit to a Hill-equation. (C) Fluorescence spectra (ex. 560 nm) with increasing [Hg²⁺]. (D) Selectivity of the probe.

FIG. 5 shows FRET-based Hg²⁺ probe. (A) Design scheme. (B) Donor channel fluorescence in the absence and presence of Hg²⁺. The donor channel fluorescence of HgA2-T is indistinguishable from that of the buffer and is, therefore, omitted from the graph.

FIG. 6 the FIT-aptamer for Ag⁺ (AgA). (A) Design scheme. Only bases involved in binding to Ag⁺ are shown for clarity. (B) Fluorescence enhancement vs [Ag⁺]. (C) Selectivity of the probe.

FIG. 7 shows the detecting of metallo-bps within a duplexed strand. (A) Design scheme with T-Hg²⁺-T as an example. (B) Fluorescence enhancement when Hg²⁼ is added to DNA duplexes with 0, 1, and 2 T-T mismatches adjacent to the dye. The first column shows that the dye-labeled strands do not turn on in the presence of Hg²⁺ in their single-stranded forms.

FIG. 8 shows fluorescence enhancement upon T-Hg²⁺-T base pairing within a duplexed strand.

FIG. 9 shows the FIT-aptamer (I-mD) for pH sensing. (A) Design scheme. Only bases involved in i-motif formation are shown for clarity. (B) Circular dichroism spectra showing i-motif formation at lower pH. (C) Fluorescence enhancement (I_{f}/I₀) vs pH. Dashed line indicates theoretical fit to a sigmoidal curve.

FIG. 10 shows the FIT-aptamers for two epitope binding. (A) Design scheme. (B) Fluorescence enhancement vs concentration of thrombin denoted as [THR]. (C) Selectivity of the probe.

FIG. 11 shows (A) A scheme of two epitope binding aptamers for thrombin that incorporate a FRET pair. (B) Excitation/emission spectra of THR1-F and THR2-T. (C) FRET efficiency of THR1-F and THR2-T as a function of thrombin concentration. (D) Fluorescence spectra of THR1-F and THR2-T for different concentrations of thrombin. (E) Fluorescence spectra of FIT-based strategy for different thrombin concentrations.

FIG. 12 shows the fluorescence response of 50 nM THR1 D and 50 nM THR2 to varying amounts of thrombin in a buffer/serum mixture.

FIG. 13 shows the FIT-based split-aptamer. (A) Design scheme. (B) The fluorescence response of 50 nM Split-THRa and 50 nM Split-THRb to increasing concentrations of thrombin. (C) Selectivity of the probe.

FIG. 14 shows the ¹H NMR (400 MHz, DMSO-d6, 298 K) spectrum of quinoline blue derivative D.

FIG. 15 shows (A) effect of pH and (B) ionic strength on fluorescence enhancement.

FIG. 16 shows the CD spectra of (A) HgA1, (B) HgA1+HgA1comp, (C) HgA2, and (D) AgA in the presence and absence of Hg²⁺/Ag⁺.

FIG. 17 shows two epitope aptamer binding to THR. (A) Effect of temperature on fluorescence enhancement. (B) Spectra showing fluorescence enhancement upon thrombin addition to the probes at 10° C. (ex. 560 nm). (C) Fluorescence spectrum of 100 nM thrombin (blue) in comparison to the spectra of the probes in the absence (black) and presence (red) of 100 nM thrombin (ex. 560 nm).

FIG. 18 depicts the concept for the use of FIT-Probes in a high-throughput screen for novel metallo-base pairs and shows potential metallo-base pairs for Hg(II) screening.

FIG. 19 shows potential metallo-base pairs for Ag(I) screening.

FIG. 20 shows potential metallo-base pairs for Cu(II) screening.

FIG. 21 shows potential metallo-base pairs for Mn(II) screening.

FIG. 22 shows potential metallo-base pairs for Zn(II) screening.

FIG. 23 shows potential metallo-base pairs for Au(III) screening.

### DETAILED DESCRIPTION

Aptamers are oligonucleotide sequences which can be evolved to bind to various analytes of interest. Provided herein is a general design strategy that transduces an aptamer-target binding event into a fluorescence readout via the use of a viscosity-sensitive dye. Target binding to the aptamer leads to restriction of the internal rotation of the dye (e.g., through forced intercalation (FIT) of the dye between oligonucleotide base pairs), thereby increasing its fluorescence. Specifically, it is demonstrated herein that FIT-aptamers can report target presence through, for example and without limitation, intramolecular conformational changes, sandwich assays, as well as target-templated reassociation of split-aptamers, showing that the most common aptamer-target binding modes can be coupled to a FIT-based readout. In some embodiments, this strategy is used to detect the formation of a metallo-base pair within a duplexed strand and is therefore attractive for screening for metal-mediated base pairing events. Importantly, FIT-aptamers reduce false-positive signals typically associated with fluorophore-quencher based systems, quantitatively outperform FRET-based probes by providing up to 15-fold higher signal to background ratios, and allow rapid and highly sensitive target detection (nanomolar range) in complex media such as human serum. Taken together, FIT-aptamers are a new class of signaling aptamers which contain a single modification, yet can be used to detect a broad range of targets.

An “aptamer” as used herein is an oligonucleotide sequence that binds to a target analyte.

As used herein, a “FIT aptamer” is an aptamer that comprises a detectable marker situated at an internal location within the aptamer. Thus, it will be understood that any feature of an aptamer (e.g., length, type (DNA, RNA, modified forms thereof)) described herein is also applicable to a FIT aptamer.

As used in this specification and the appended claims, the singular forms “a,” “an,” and “the” include plural reference unless the context clearly dictates otherwise.

The terms “polynucleotide” and “oligonucleotide” are interchangeable as used herein.

As used herein, a “non-canonical base pair” is any base pair that is not formed through standard Watson-Crick base pairing.

As used herein, “duplex” refers to a region in two complementary or sufficiently complementary oligonucleotides that form base pairs with one another, either by Watson-Crick base pairing or any other manner that allows for a stabilized duplex between oligonucleotide strands that are complementary or sufficiently complementary. “Sufficiently complementary” refers to the degree of complementarity between two nucleotide sequences such that a stable duplex is formed under the conditions in which the duplex is used. In various embodiments, sufficiently complementary nucleotide sequences are sequences that are or are at least 50%, 55%, 60%, 65%, 70%, 75%, 80%, 85%, 90%, 95%, or 99% complementary within a duplex. In some embodiments, sufficiently complementary nucleotide sequences are sequences that are 100% complementary within a duplex. In further embodiments, two nucleotide sequences are sufficiently complementary when there are 1, 2, 3, 4, 5, 6, 7, 8, 9, 10 or more mismatches between the two nucleotide sequences.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Aptamers

The present disclosure is directed to aptamers, such as FIT aptamers, as well as methods of making and using the aptamers. In some aspects, the disclosure provides a composition comprising a FIT aptamer as described herein. In various embodiments, an aptamer is a DNA oligonucleotide or a modified form thereof, an RNA oligonucleotide or a modified form thereof, or a combination thereof. Aptamers may be single stranded, double stranded, or partially double stranded. As described herein, a FIT aptamer is an aptamer that comprises a detectable marker situated at an internal location within the aptamer. In various embodiments, the detectable marker is situated at a position that is 1, 2, 3, 4, 5, 6, 7, 8, 9, 10, or more nucleotides from a terminus (i.e., 5′ or 3′ terminus) of the aptamer. In some embodiments, the detectable marker is situated at a position that is x nucleotides from a terminus (i.e., 5′ or 3′ terminus) of the aptamer, wherein x is an integer that is 1, n/2, or any integer between 1 and n/2, wherein n is (i) the length of the aptamer and (ii) an even number. In further embodiments, the detectable marker is situated at a position that is x nucleotides from a terminus (i.e., 5′ or 3′ terminus) of the aptamer, wherein x is an integer that is 1, (n+1)/2, or any integer between 1 and (n+1)/2, wherein n is (i) the length of the aptamer and (ii) an odd number. In some embodiments, the detectable marker is situated at about a midpoint along the length of the aptamer, wherein the nucleotide sequences on either side of the detectable marker are sufficiently complementary to form a duplex. In any of the aspects or embodiments of the disclosure, the FIT aptamer consists of one detectable marker. In some embodiments, the FIT aptamer comprises 1, 2, 3, 4, 5, 6, 7, 8, 9, 10, or more detectable markers.

Oligonucleotides. The disclosure contemplates, in any aspects or embodiments described herein, the use of DNA oligonucleotides, RNA oligonucleotides, or combinations of the two types. In any aspects or embodiments described herein, an oligonucleotide is single-stranded, double-stranded, or partially double-stranded. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and pypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═(including R⁵ when used as a linkage to a succeeding monomer), —CH₂—O—NR^{H}—, —CO—NR^{H}—CH₂—, —CH₂—NR^{H}—O—, —CH₂—NR^{H}—O—CO—O—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an aptamer of the disclosure, or a modified form thereof, is generally about 10 nucleotides to about 100 nucleotides in length. More specifically, an aptamer of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all aptamers intermediate in length of the sizes specifically disclosed to the extent that the aptamer is able to achieve the desired result. In further embodiments, an aptamer of the disclosure is about 5 nucleotides to about 1000 nucleotides in length. In further embodiments, an aptamer of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all aptamers intermediate in length of the sizes specifically disclosed to the extent that the aptamer is able to achieve the desired result. Accordingly, in various embodiments, an aptamer of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an aptamer of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. It will be understood that, when more than one aptamer is used together (e.g., in a sandwich assay) each aptamer may be a different length, or some or all of the aptamers may be the same length. It will be appreciated that oligonucleotides of any of the foregoing lengths are also contemplated by the disclosure.

Methods of attaching detectable markers and additional moieties as described herein to an oligonucleotide are known in the art.

Spacers. In some aspects and embodiments, one or more aptamers or oligonucleotides comprise a spacer. “Spacer” as used herein means a moiety that serves to increase or provide distance between portions of the aptamer. Thus, spacers are contemplated being located between a portion of an aptamer (such as a FIT aptamer) that binds to a target analyte and a portion that binds to another portion of the same aptamer or to a portion of a different aptamer.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an aptamer comprises 1, 2, 3, 4, 5, or more spacer (e.g., Spacer-18 (hexaethyleneglycol)) moieties. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the aptamer to perform an intended function (e.g., bind to a target analyte). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

### Detectable Markers

In any of the aspects or embodiments of the disclosure, a FIT aptamer comprises a detectable marker. In various embodiments, the FIT aptamer comprises 2, 3, 4, 5, 6, 7, 8, 9, 10, or more detectable markers, which may be either all the same or one or more detectable markers may be different. In various embodiments, the one or more detectable markers is situated at any internal position with an aptamer/oligonucleotide. As used herein, a “detectable marker” is a marker that exhibits internal rotation-dependent fluorescence or is viscosity-sensitive. In various embodiments, the detectable marker is thiazole orange (TO), quinoline blue, quinoline violet, thiazole red, a derivative thereof, or a cyanine derivative. In various embodiments, however, a FIT aptamer further comprises an additional moiety, wherein the additional moiety is a fluorophore that does not exhibit internal rotation-dependent fluorescence.

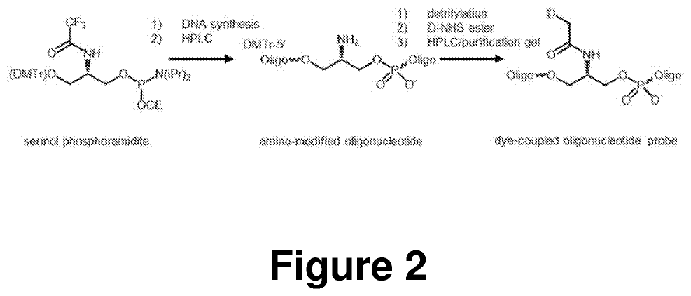

Methods of attaching a detectable marker to an aptamer are known in the art and exemplified herein (see, e.g., Example 2 and FIG. 2). In some embodiments, the detectable marker is situated at a position that is x nucleotides from a terminus (i.e., 5′ or 3′ terminus) of an aptamer, wherein x is an integer that is 1, n/2, or an integer between 1 and n/2, and n is (i) the length of the aptamer and (ii) an even number. In further embodiments, one or more detectable markers are situated at different positions within an aptamer, with each detectable marker being situated at a position that is x nucleotides from a terminus (i.e., 5′ or 3′ terminus) of the aptamer, wherein x is an integer that is 1, n/2, or an integer between 1 and n/2, and n is (i) the length of the aptamer and (ii) an even number. In some embodiments, the detectable marker is situated at a position that is x nucleotides from a terminus (i.e., 5′ or 3′ terminus) of an aptamer, wherein x is an integer that is 1, (n+1)/2 or any integer between 1 and (n+1)/2, and n is (i) the length of the aptamer and (ii) an odd number. In further embodiments, one or more detectable markers are situated at different positions within an aptamer, with each detectable marker being situated at a position that is x nucleotides from a terminus of the aptamer, wherein x is an integer that is 1, (n+1)/2 or any integer between 1 and (n+1)/2, and n is (i) the length of the aptamer and (ii) an odd number.

### Additional Moieties

The disclosure contemplates, in various embodiments, that an aptamer of the disclosure (such as a FIT aptamer) comprises an additional moiety such as, without limitation, a targeting agent, a therapeutic agent, an additional fluorophore that is a detectable marker or that does not exhibit internal rotation-dependent fluorescence, or a combination thereof. Methods of attaching additional moieties to an aptamer are known in the art. In some embodiments, an oligonucleotide comprises an additional moiety.

Examples of the additional fluorophores include without limitation a FRET pair, AlexaFluor dyes, ATTO dyes, BAPTA derivatives, fluorescein derivatives, rhodamine derivatives, and coumarin derivatives.

Examples of targeting moieties contemplated by the disclosure include without limitation proteins, peptides, oligonucleotides, lipids, carbohydrates, small molecules, and chemical functional groups that can be used to direct the FIT aptamers to specific cell types, specific sub-cellular compartments, or specific organs.

Examples of therapeutic agents contemplated by the disclosure include without limitation therapeutic small molecules (e.g., anti-cancer drugs, anti-diabetes drugs), protein or peptide-based drugs, therapeutic oligonucleotides (e.g., immunomodulatory, gene-regulatory), or a combination thereof.

### Methods of Using FIT Aptamers

The present disclosure is directed to aptamers, oligonucleotide sequences that can bind to an analyte of interest, to study various targets. The disclosure offers new signal transduction reagents and methods for target presence readout. Accordingly, the disclosure provides FIT aptamers that are useful for detecting presence of a target analyte through, for example and without limitation, intramolecular conformational changes, sandwich assays, target-templated reassociation of split-aptamers, and identification of non-canonical base pairs, each as described herein. It is contemplated that most aptamer-target binding modes known in the art can be coupled to a FIT-based readout.

Intramolecular conformational changes. As described herein, FIT aptamers comprise a detectable marker situated at an internal location within the aptamer. In some aspects, the disclosure provides a method of detecting the presence of a target analyte comprising the step of contacting the target analyte with an aptamer comprising a detectable marker situated at an internal location within the aptamer, wherein the contacting results in binding of the target analyte to the aptamer, wherein target analyte binding to the aptamer results in restriction of internal rotation of the marker, resulting in a detectable change in the marker. In some embodiments, target analyte binding to the aptamer results in forced intercalation (FIT) of the marker between oligonucleotide base pairs of the aptamer. In some embodiments, target analyte binding to the aptamer results in intramolecular duplex formation in the aptamer. In further embodiments, target analyte binding to the aptamer results in triplex or tetraplex formation in the aptamer.

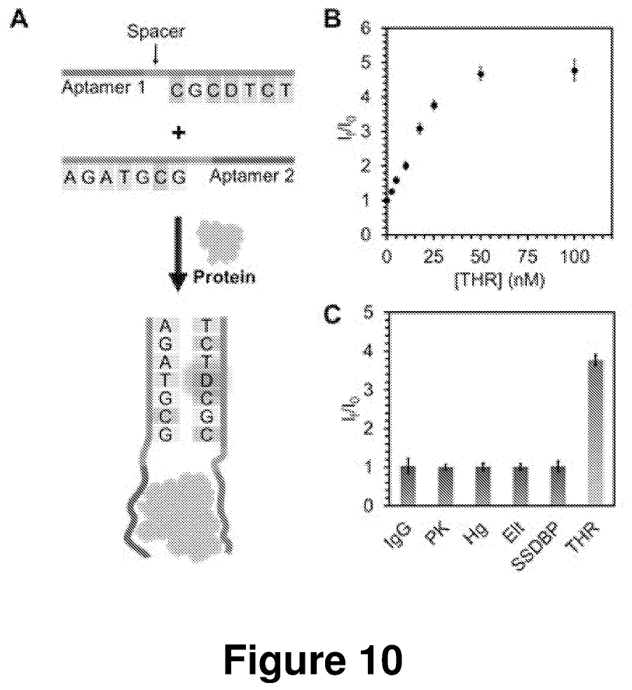

Sandwich Assays/Split Aptamers. A sandwich assay generally refers to the use of more than one aptamer to bind to a target analyte, wherein each of the more than one aptamers binds to a different binding site on the target analyte. Thus, in some embodiments, a target analyte having two or more binding sites is contacted with two different aptamers that bind independently to different binding sites on the target. See, e.g., FIG. 10A. Accordingly, in some aspects, the disclosure provides a method of detecting the presence of a target analyte comprising the step of contacting the target analyte with (a) an aptamer or portion thereof comprising (i) nucleotide sequence X, (ii) nucleotide sequence Y which binds to the target analyte, either alone or in combination with nucleotide sequence Y′ and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change in the marker. In some embodiments, nucleotide sequence Y and nucleotide sequence Y′ bind to different binding sites of the target analyte.

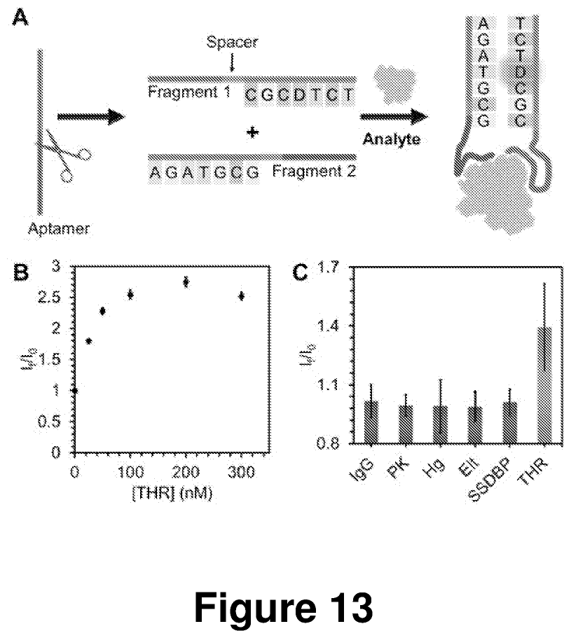

In some aspects, methods of the disclosure include the use of split aptamers. In some embodiments, it is contemplated that a target analyte has only a single binding site such that it may be bound by only a single aptamer. Split aptamer methods involve the use of a single aptamer sequence that is split to create two aptamer oligonucleotides, wherein each aptamer oligonucleotide comprises a portion that binds to the single binding site on the target analyte. The single aptamer may be split into two portions and additional nucleotides appended to each of the two portions, wherein the appended nucleotide sequences are sufficiently complementary to hybridize to each other. See, e.g., FIG. 13A. Accordingly, in some aspects the disclosure provides a method of detecting the presence of a target analyte comprising the step of contacting the target analyte with (a) an aptamer or portion thereof comprising (i) nucleotide sequence X, (ii) nucleotide sequence Y which binds to the target analyte, either alone or in combination with nucleotide sequence Y′ and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change in the marker. In some embodiments, nucleotide sequence Y and nucleotide sequence Y′ together bind to the same binding site of the target analyte. In some embodiments, nucleotide sequence Y will not bind to the target analyte in the absence of nucleotide sequence Y′ also binding to the target analyte. In some embodiments, binding to the target analyte requires both portions of the aptamer.

Non-canonical base pair identification. In some aspects, the disclosure provides a method of identifying a non-canonical base pair comprising the step of contacting an ion with (a) a first oligonucleotide comprising a detectable marker situated at an internal location within the first oligonucleotide and (b) a second oligonucleotide, wherein the first oligonucleotide and the second oligonucleotide are sufficiently complementary to hybridize to each other and form a duplex, but are not complementary at a position immediately adjacent to the detectable marker when the first oligonucleotide is hybridized to the second oligonucleotide; and wherein binding of the ion to the duplex results in restriction in the internal rotation of the marker, resulting in a detectable change in the marker and thereby identifying the non-canonical base pair. In some embodiments, the first oligonucleotide and the second oligonucleotide are sufficiently complementary to hybridize to each other and form a duplex, but the duplex comprises a single nucleotide mismatch at a position immediately adjacent to the detectable marker when the first oligonucleotide is hybridized to the second oligonucleotide. In further embodiments, the first oligonucleotide and the second oligonucleotide are sufficiently complementary to hybridize to each other and form a duplex, but the duplex comprises a nucleotide mismatch on both sides of the detectable marker when the first oligonucleotide is hybridized to the second oligonucleotide. In various embodiments, the first oligonucleotide is or is at least 75%, 80%, 85%, 90%, 95%, or 99% complementary to the second oligonucleotide.

In further aspects, the disclosure provides a method of identifying a non-canonical base pair comprising the step of contacting an ion with an aptamer comprising a detectable marker situated at an internal location within the aptamer, wherein the aptamer is able to form an intramolecular duplex, and the duplex comprises a nucleotide mismatch at a position immediately adjacent to the detectable marker; and wherein binding of the ion to the duplex results in restriction in the internal rotation of the marker, resulting in a detectable change in the marker and thereby identifying the non-canonical base pair. In some embodiments, the duplex comprises a single nucleotide mismatch at a position immediately adjacent to the detectable marker. In some embodiments, the duplex comprises a single nucleotide mismatch on both sides of the detectable marker.

In some embodiments, binding of the ion to the duplex results in forced intercalation (FIT) of the marker in the duplex. In some embodiments, the detectable marker is a marker with internal rotation-dependent fluorescence. In some embodiments, the detectable marker with internal rotation-dependent fluorescence is a viscosity-sensitive marker. In further embodiments, the detectable marker is thiazole orange (TO), quinoline blue, quinoline violet, thiazole red, a derivative thereof, or a cyanine derivative. In some embodiments, the ion is a cation. In some embodiments, the ion is a metal ion. In some embodiments, the metal ion is a mercury ion, a copper ion, a silver ion, zinc ion, gold ion, manganese ion, or a combination thereof. In some embodiments, the ion is an anion.

### Target Analytes

In various aspects and embodiments, the disclosure provides methods of detecting target analytes. Target analytes contemplated by the disclosure include without limitation a protein, an ion, a small molecule, a lipid, a carbohydrate, an oligosaccharide, a cell, an oligonucleotide, or a combination thereof.

In some embodiments, the ion is an anion or a cation. In some embodiments, ions contemplated by the disclosure are metal ions. In further embodiments, the metal ion is a mercury ion, a copper ion, a silver ion, zinc ion, gold ion, manganese ion, or a combination thereof. In some embodiments, the ion is a hydrogen ion. In further embodiments, the change in the detectable marker is indicative of a pH change.

Proteins are understood in the art and may be either naturally occurring or non-naturally occurring. Naturally occurring proteins include without limitation biologically active proteins (including antibodies) that exist in nature or can be produced in a form that is found in nature by, for example, chemical synthesis or recombinant expression techniques. Naturally occurring proteins also include lipoproteins and post-translationally modified proteins, such as, for example and without limitation, glycosylated proteins. Non-naturally occurring proteins contemplated by the present disclosure include but are not limited to synthetic proteins, as well as fusion proteins.

Proteins include antibodies along with fragments and derivatives thereof, including but not limited to Fab′ fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

The term “small molecule,” as used herein, refers to a chemical compound, or any other low molecular weight organic compound, either natural or synthetic. By “low molecular weight” is meant compounds having a molecular weight of less than 1000 Daltons, typically between 100 and 700 Daltons.

In some embodiments, the target analyte is an oligonucleotide. In such embodiments, it is contemplated that target oligonucleotide binding to a FIT aptamer duplex forms a “triplex structure,” causing a conformational change in the detectable marker that results in detection of the marker.

Lipids are understood in the art. Non-limiting examples include tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, polyunsaturated sterols of different lengths, saturation states, saturated C8-C22 fatty acids, saturated C8-C22 ether derivatives of glycerol, saturated and unsaturated amide derivatives of C8-C22 fatty acids and mono- and 1,2- or 1, 3-di-amino glycerols, derivatives thereof, or a combination thereof.

Carbohydrates are known in the art. Non-limiting examples include sucrose, xylose, mannose, fructose, maltose, lactose, galactose, derivatives thereof, or a combination thereof.

Oligosaccharides are understood in the art. Non-limiting examples include cellobiose, cellodextrin, B-cyclodextrin, indigestible dextrin, gentio-oligosaccharide, gluco-oligosaccharide, isomaltoligosaccharide, isomaltose, isomatriose, panose, leucrose), Palatinose, cyananderose, D-agatose, D-lyxo-hexulose, lactosucrose, α-galactooligosaccharide, β-galactooligosaccharide, Transgalactooligosaccharides, lactulose, 4′-galatosyllactose, synthetic galactooligosaccharides, fructans-Levan-type, frustans-Inutin-type, 1f-β-fructofuranosylnystose (1f-β-fructofuranosylnystose), xylooligosaccharide, raffinose (lafinose), lactosucrose and arabino-oligosaccharides, derivatives thereof, or a combination thereof.

### REFERENCES

(1) Ellington, A. D.; Szostak, J. W. In Vitro Selection of RNA Molecules That Bind Specific Ligands. Nature 1990, 346, 818-822.
(2) Tuerk, C.; Gold, L. Systematic Evolution of Ligands by Exponential Enrichment: RNA Ligands to Bacteriophage T4 DNA Polymerase. Science 1990, 249, 505-510.
(3) Keefe, A. D.; Pai, S.; Ellington, A. Aptamers as Therapeutics. Nat. Rev. Drug Discov. 2010, 9, 537-550.
(4) Brody, E. N.; Gold, L. Aptamers as Therapeutic and Diagnostic Agents. J. Biotechnol. 2000, 74, 5-13.
(5) Liu, J.; Cao, Z.; Lu, Y. Functional Nucleic Acid Sensors. Chem. Rev. 2009, 109, 1948-1998.
(6) Nutiu, R.; Li, Y. Structure-Switching Signaling Aptamers. J. Am. Chem. Soc. 2003, 125, 4771-4778.
(7) Wickramaratne, T. M.; Pierre, V. C. Turning an Aptamer into a Light-Switch Probe with a Single Bioconjugation. Bioconjugate Chem. 2015, 26, 63-70.
(8) Hamaguchi, N.; Ellington, A.; Stanton, M. Aptamer Beacons for the Direct Detection of Proteins. Anal. Biochem. 2001, 294, 126-131.
(9) Tang, Z.; Mallikaratchy, P.; Yang, R.; Kim, Y.; Zhu, Z.; Wang, H.; Tan, W. Aptamer Switch Probe Based on Intramolecular Displacement. J. Am. Chem. Soc. 2008, 130, 11268-11269.
(10) Modi, S.; Swetha, M. G.; Goswami, D.; Gupta, G. D.; Mayor, S.; Krishnan, Y. A DNA Nanomachine That Maps Spatial and Temporal PH Changes inside Living Cells. Nat. Nanotechnol. 2009, 4, 325-330.
(11) Paige, J. S.; Wu, K. Y.; Jaffrey, S. R. RNA Mimics of Green Fluorescent Protein. Science 2011, 333, 642-646.
(12) Paige, J. S.; Nguyen-Duc, T.; Song, W.; Jaffrey, S. R. Fluorescence Imaging of Cellular Metabolites with RNA. Science 2012, 335, 1194.
(13) Warner, K. D.; Sjekloa, L.; Song, W.; Filonov, G. S.; Jaffrey, S. R.; Ferré-D'Amaré, A. R. A Homodimer Interface without Base Pairs in an RNA Mimic of Red Fluorescent Protein. Nat. Chem. Biol. 2017, 13, 1195-1201.
(14) Song, W.; Filonov, G. S.; Kim, H.; Hirsch, M.; Li, X.; Moon, J. D.; Jaffrey, S. R. Imaging RNA Polymerase III Transcription Using a Photostable RNA-Fluorophore Complex. Nat. Chem. Biol. 2017, 13, 1187-1194.
(15) Zhou, W.; Ding, J.; Liu, J. 2-Aminopurine-Modified DNA Homopolymers for Robust and Sensitive Detection of Mercury and Silver. Biosens. Bioelectron. 2017, 87, 171-177.
(16) Wang, Z.; Heon Lee, J.; Lu, Y. Highly Sensitive “Turn-on” Fluorescent Sensor for Hg²⁺ in Aqueous Solution Based on Structure-Switching DNA. Chem. Commun. 2008, 7, 6005-6007.
(17) Yang, Y.; Huang, J.; Yang, X.; Quan, K.; Wang, H.; Ying, L.; Xie, N.; Ou, M.; Wang, K. FRET Nanoflares for Intracellular MRNA Detection: Avoiding False Positive Signals and Minimizing Effects of System Fluctuations. J. Am. Chem. Soc. 2015, 137, 8340-8343.
(18) Leavesley, S. J.; Rich, T. C. Overcoming Limitations of FRET Measurements. Cytometry A 2016, 89, 325-327.
(19) Filonov, G. S.; Moon, J. D.; Svensen, N.; Jaffrey, S. R. Broccoli: Rapid Selection of an RNA Mimic of Green Fluorescent Protein by Fluorescence-Based Selection and Directed Evolution. J. Am. Chem. Soc. 2014, 136, 16299-16308.
(20) Kohler, O.; Jarikote, D. V.; Seitz, O. Forced Intercalation Probes (FIT Probes): Thiazole Orange as a Fluorescent Base in Peptide Nucleic Acids for Homogeneous Single-Nucleotide-Polymorphism Detection. ChemBioChem 2005, 6, 69-77.
(21) Hövelmann, F.; Gaspar, I.; Chamiolo, J.; Kasper, M.; Steffen, J.; Ephrussi, A.; Seitz, O. LNA-Enhanced DNA FIT-Probes for Multicolour RNA Imaging. Chem. Sci. 2016, 7, 128-135.
(22) Hövelmann, F.; Bethge, L.; Seitz, O. Single Labeled DNA FIT Probes for Avoiding False-Positive Signaling in the Detection of DNA/RNA in QPCR or Cell Media. ChemBioChem 2012, 13, 2072-2081.
(23) Hövelmann, F.; Gaspar, I.; Ephrussi, A.; Seitz, O. Brightness Enhanced DNA FIT-Probes for Wash-Free RNA Imaging in Tissue. J. Am. Chem. Soc. 2013, 135, 19025-19032.
(24) Miyake, Y.; Togashi, H.; Tashiro, M.; Yamaguchi, H.; Oda, S.; Kudo, M.; Tanaka, Y.; Kondo, Y.; Sawa, R.; Fujimoto, T.; et al. MercuryII-Mediated Formation of Thymine-HgII-Thymine Base Pairs in DNA Duplexes. J. Am. Chem. Soc. 2006, 128, 2172-2173.
(25) US EPA, O. Drinking Water Contaminants—Standards and Regulations. Drink. Water Contam. 2013.
(26) Kondo, J.; Tada, Y.; Dairaku, T.; Hattori, Y.; Saneyoshi, H.; Ono, A.; Tanaka, Y. A Metallo-DNA Nanowire with Uninterrupted One-Dimensional Silver Array. Nat. Chem. 2017, 9, 956-960.
(27) Schmidt, O. P.; Mata, G.; Luedtke, N. W. Fluorescent Base Analogue Reveals T-Hg^{II}-T Base Pairs Have High Kinetic Stabilities That Perturb DNA Metabolism. J. Am. Chem. Soc. 2016, 138, 14733-14739.
(28) Walter, N. G.; Harris, D. A.; Pereira, M. J. B.; Rueda, D. In the Fluorescent Spotlight: Global and Local Conformational Changes of Small Catalytic RNAs. Biopolymers. 2001, 61, 224-242.
(29) Song, D.; Graham, T. G. W.; Loparo, J. J. A General Approach to Visualize Protein Binding and DNA Conformation without Protein Labelling. Nat. Commun. 2016, 7, 10976.
(30) Benevides, J. M.; Thomas, G. J. Local Conformational Changes Induced in B-DNA by Ethidium Intercalation. Biochemistry 2005, 44, 2993-2999.
(31) Bradrick, T. D.; Marino, J. P. Ligand-Induced Changes in 2-Aminopurine Fluorescence as a Probe for Small Molecule Binding to HIV-1 TAR RNA. RNA 2004, 10, 1459-1468.
(32) Kaul, M.; Barbieri, C. M.; Pilch, D. S. Defining the Basis for the Specificity of Aminoglycoside-rRNA Recognition: A Comparative Study of Drug Binding to the A Sites of Escherichia Coli and Human rRNA. J. Mol. Biol. 2005, 346, 119-134.
(33) Fourmy, D.; Yoshizawa, S.; Puglisi, J. D. Paromomycin Binding Induces a Local Conformational Change in the A-Site of 16 S rRNA. J. Mol. Biol. 1998, 277, 333-345.
(34) Zeraati, M.; Langley, D. B.; Schofield, P.; Moye, A. L.; Rouet, R.; Hughes, W. E.; Bryan, T. M.; Dinger, M. E.; Christ, D. I-Motif DNA Structures Are Formed in the Nuclei of Human Cells. Nat. Chem. 2018, 10, 631-637.
(35) Nesterova, I. V.; Nesterov, E. E. Rational Design of Highly Responsive PH Sensors Based on DNA I-Motif. J. Am. Chem. Soc. 2014, 136, 8843-8846.
(36) Macbeath, G. Protein Microarrays and Proteomics. Nat. Genet. 2002, 32, 526-532.
(37) Selvarajah, S.; Negm, O. H.; Hamed, M. R.; Tubby, C.; Todd, I.; Tighe, P. J.; Harrison, T.; Fairclough, L. C. Development and Validation of Protein Microarray Technology for Simultaneous Inflammatory Mediator Detection in Human Sera. Mediators Inflamm. 2014, 2014, 820304.
(38) Onopiuk, A.; Tokarzewicz, A.; Gorodkiewicz, E. Cystatin C: A Kidney Function Biomarker. Adv. Clin. Chem. 2015, 68, 57-69.
(39) Heyduk, E.; Heyduk, T. Nucleic Acid-Based Fluorescence Sensors for Detecting Proteins. Anal. Chem. 2005, 77, 1147-1156.
(40) Bock, L. C.; Griffin, L. C.; Latham, J. A.; Vermaas, E. H.; Toole, J. J. Selection of Single-Stranded DNA Molecules That Bind and Inhibit Human Thrombin. Nature 1992, 355, 564-566.
(41) Tasset, D. M.; Kubik, M. F.; Steiner, W. Oligonucleotide Inhibitors of Human Thrombin That Bind Distinct Epitopes. J. Mol. Biol. 1997, 272, 688-698.
(42) Horne III, M. K.; Merryman, P. K.; Cullinane, A. M.; Nghiem, K.; Alexander, H. R. The Impact of Major Surgery on Blood Coagulation Factors and Thrombin Generation. Am. J. Hematol. 2007, 82, 815-820.
(43) Brummel-Ziedins, K. E.; Pouliot, R. L.; Mann, K. G. Thrombin Generation: Phenotypic Quantitation. J. Thromb. Haemost. 2004, 2, 281-288.
(44) Chen, A.; Yan, M.; Yang, S. Split Aptamers and Their Applications in Sandwich Aptasensors. Trends Anal. Chem. 2016, 80, 581-593.

### EXAMPLES

### Example 1

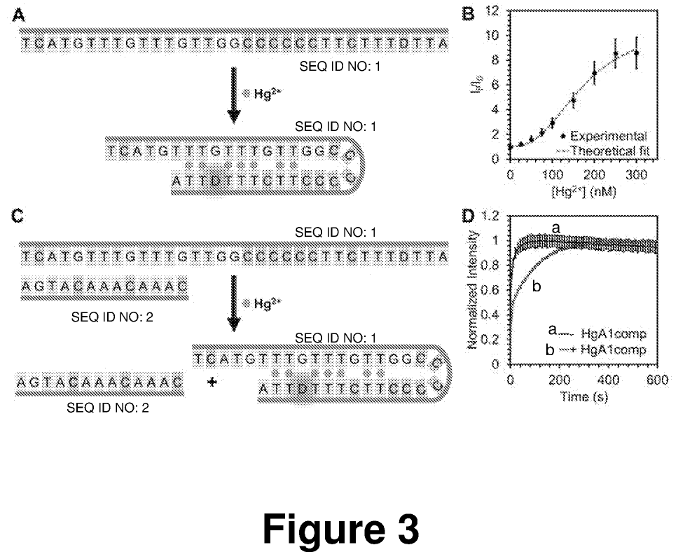

To evaluate the feasibility of realizing FIT-aptamers, a previously reported DNA sequence was chosen (Table 1), known to recognize Hg²⁺, as an example of an aptamer that binds to its target through an intramolecular conformational change.¹⁶ The aptamer sequence was used as a single-stranded probe and the FIT-dye quinoline blue (D) as a nucleobase surrogate. It was considered that this aptamer adopts a hairpin-like structure in the presence of Hg²⁺ due to the Hg²⁺-mediated bridging of thymine (T) bases (T-Hg²⁺-T).^{16,24} Therefore, it was hypothesized that if a base sandwiched between two Ts in the aptamer sequence was replaced with D, forced intercalation of D between the metallo-base pairs (bps) would turn on its fluorescence. The FIT-aptamer (HgA1) was synthesized by substituting the fourth base from the 3′ end of the sequence with an amino-modifier to which D-carboxylate was conjugated via carbodiimide crosslinking chemistry (FIGS. 2-3). HgA1 was then titrated with Hg²⁺ in a buffered solution. The fluorescence enhancement factor (I_{f}/I₀), defined as the ratio of the fluorescence in the presence of target (signal, I_{f}) to the initial fluorescence (background, I₀), increases with increasing concentrations of Hg²⁺ and reaches a maximum value of approximately 8 (FIG. 3). Significantly, when a short complementary sequence (FIG. 3C) is used to partially block the aptamer akin to a structure-switching signaling aptamer,¹⁶ the response time of the probe is 5 times slower (FIG. 3D), showing that single-stranded probes such as FIT-aptamers enable faster target detection.

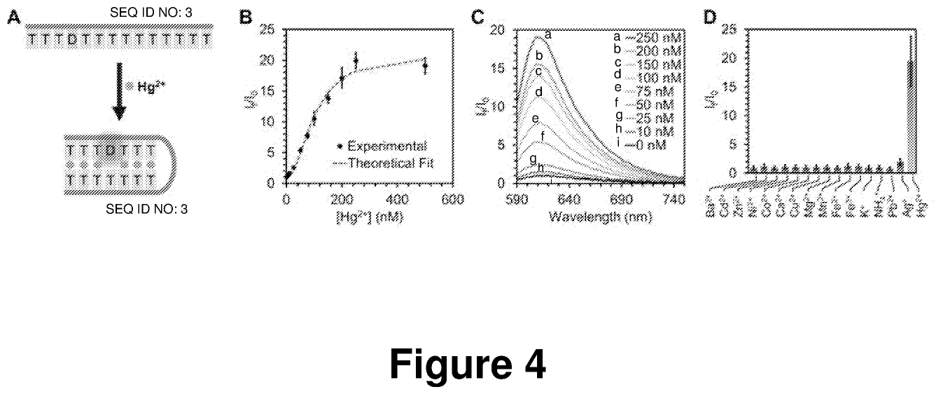

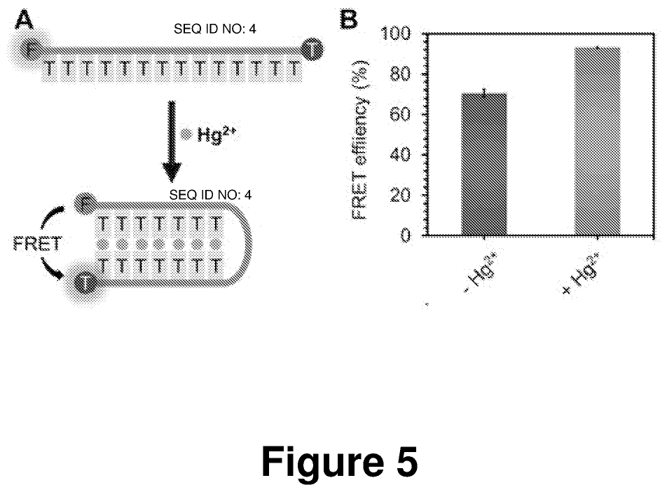

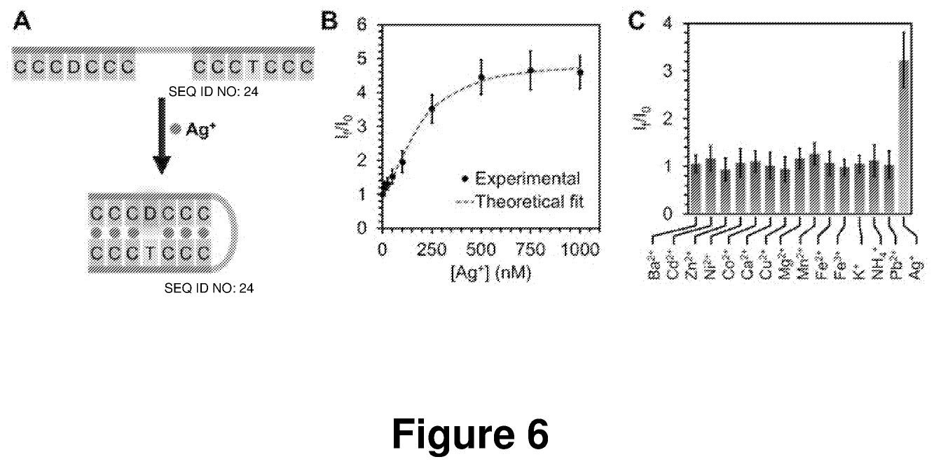

Based on these results, it was speculated that this system would enable one to create a fast, highly sensitive, and extremely simple Hg²⁺ probe. Therefore, a T₁₄ sequence was used in which the fourth base from the 5′ end was replaced with D (HgA2, FIG. 4A). Remarkably, addition of aqueous Hg²⁺ (250 nM) to HgA2 resulted in an approximately 20-fold fluorescence enhancement (FIG. 4B, C). In sharp contrast, a traditional FRET-based approach provides a signal-to-background ratio of 1.32 (FIG. 5). Based on the calibration curve of HgA2, the limit of detection (LOD) of the FIT-aptamer assay is 1.75 nM, well below the 10 nM toxicity limit for Hg²⁺ in drinking water.²⁵ Importantly, challenging the probe with a panel of 15 different metal ions yields selective turn on in the presence of Hg²⁺ (FIG. 4D). Using a similar strategy, Ag⁺ ions can be detected via the formation of C—Ag⁺—C bps using a cytosine-rich strand (FIG. 6). This is believed to be the first use of forced intercalation to measure the formation of metal-mediated non-traditional bps.

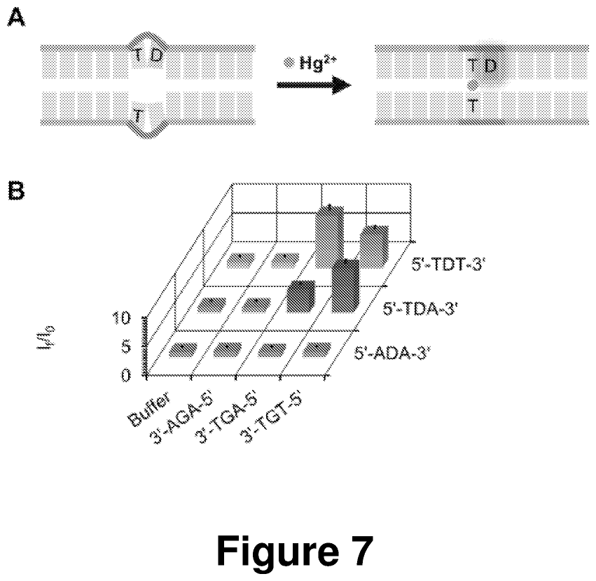

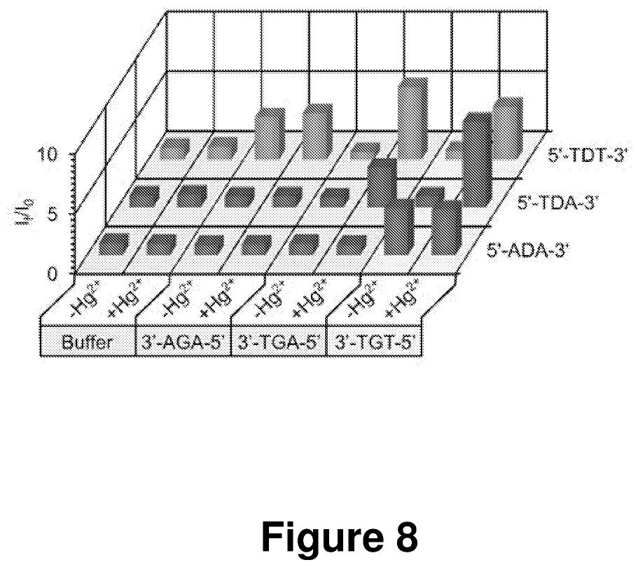

While the above examples show that FIT-aptamers can report metallo-base pair (bp) formation through global conformational changes, it was hypothesized that the sensitivity of FIT-probes to single bp mismatches²⁰ can be utilized to sense metallo-bp formation in a preformed DNA duplex. Current methods for finding metallo-bps rely primarily on single crystal X-ray diffraction studies,²⁶ which necessitate challenging DNA crystallization, or NMR,²⁴ which requires a sufficiently large number of metal binding events to distinguish resonances of metal-bound nucleobases from the inherent signal of other bases in a strand. To assess whether FIT probes can be used as a simple alternative to allow rapid detection in solution, six different 21-mer DNA sequences were synthesized: three oligonucleotides containing TDT, TDA, and ADA regions, respectively, and their complements. The sequences containing D were added to the normal sequences in all combinations to form duplexes with zero T-T, one T-T, or two T-T mismatches directly adjacent to D. It was observed that the addition of Hg²⁺ increased the fluorescence only in the duplexes where T-Hg²⁺-T bases could be formed adjacent to the dye, showing that D can report local conformational changes and, moreover, that this information can be used to determine the identity of the metallo-bp formed (FIG. 7, FIG. 8). This ability of “local probing” is not possible with FRET or fluorophore/quencher-based techniques and is useful for screening new metallo-bps, studying local structural changes in DNA and RNA during various biological processes,²⁷⁻²⁹ or identifying drug binding sites on DNA and RNA.³⁰⁻³³

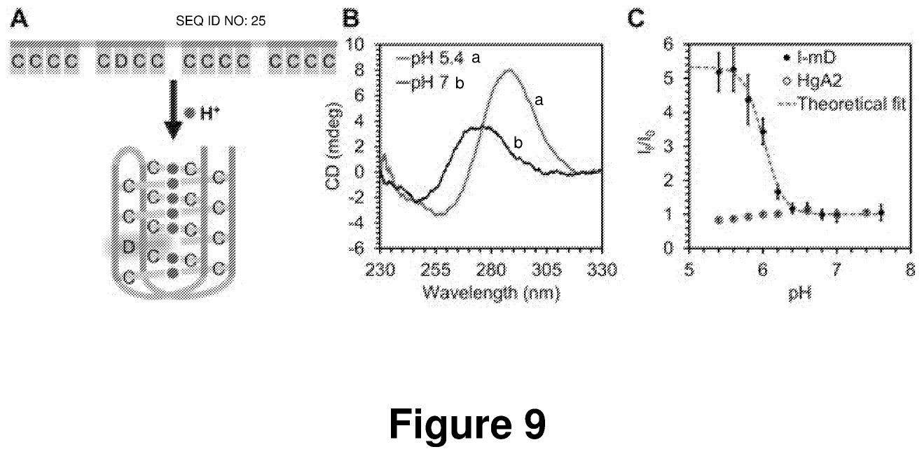

The possibility of designing FIT-aptamers that undergo more complex structural transitions upon target binding was next investigated by studying the i-motif as an example. The i-motif is a cytosine-rich “proton aptamer” that adopts a quadruplex structure at acidic pH due to the formation of C—H⁺—C bonds.^{34,35} The ninth base of the i-motif was replaced with D (I-mD, FIG. 9A). Circular dichroism spectra confirmed that the presence of D does not impede i-motif formation (FIG. 9B). Lowering the pH from 7.0 to 5.6 results in a 5-fold fluorescence enhancement (FIG. 9C). Importantly, the fluorescence of HgA2 did not significantly change within this pH range, confirming that the enhancement was due to the forced intercalation of D in the i-motif structure as opposed to the dye's inherent pH-sensitivity. Taken together, these results showed that a FIT-based strategy could be adapted to detect various analytes that cause conformational changes in aptamers.

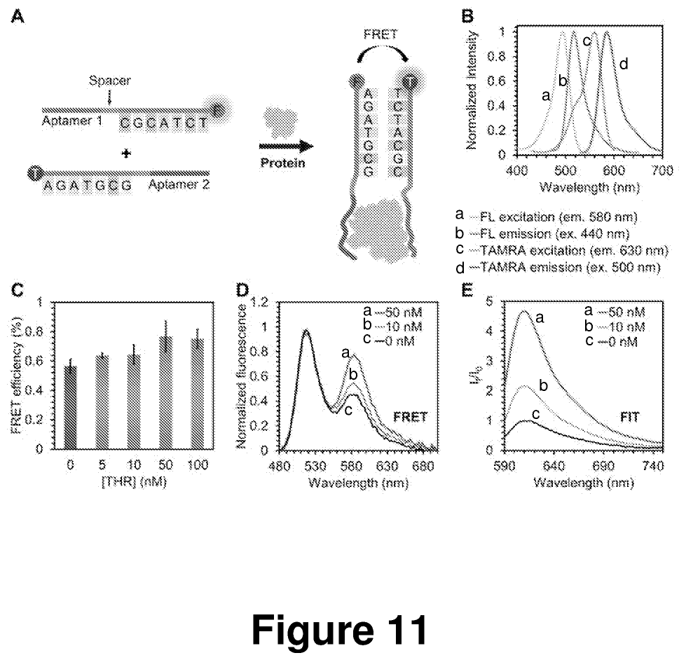

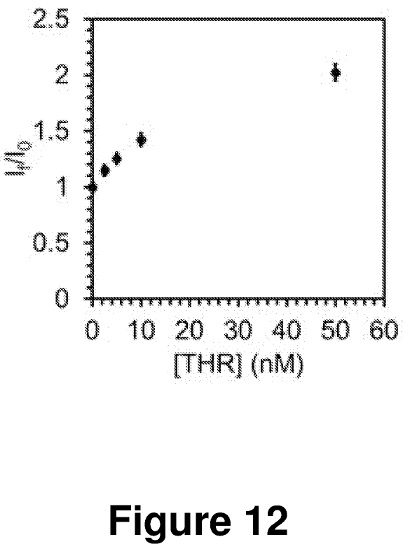

As a measure of versatility, how FIT-aptamers can be designed in alternate detection contexts was further explored. It was considered that sandwich assays for detecting proteins based on antibodies are popular due to increased specificity.³⁶⁻³⁸ Therefore, systems that require the binding of two aptamers to generate a signal output was examined.³⁹ Thrombin was used as a model system because there are known aptamers that bind to two distinct sites.^{40,41} Applying the FIT strategy, the two aptamer sequences were appended with spacer groups and short complementary sequences (FIG. 10A), one of which (THR1 D) was modified with D. The independent binding of these two aptamers (THR1D and THR2) to thrombin brings the complementary sequences into proximity, increasing their local concentration and allowing them to hybridize. In buffer, a 5-fold fluorescence enhancement upon target binding was observed (FIG. 101B). Negative controls involving proteins other than thrombin generated negligible signal (FIG. 10C). The LOD was 1.42 nM. In comparison, a FRET-based method only yielded a 1.25-fold enhancement (FIG. 11). For FIT-aptamers to be useful, it is important to be able to use them in complex media at disease relevant target concentrations. As a clinically applicable model, the potential for detecting thrombin in human serum was tested using THR1D and THR2. The results (FIG. 12) showed a limit of detection of 6.8 nM in whole serum. This value is below the 10 nM thrombin concentration in serum that is associated with blood clot formation,^{42,43} demonstrating assay utility in medical diagnostic relevant media.

As a structurally similar but distinct detection mode, how split-aptamers can be interfaced with a FIT-based strategy was also investigated. In this case, the aptamer sequence was divided into two fragments such that the presence of the target templates their re-association.⁴⁴ Utilizing thrombin, it was validated that FIT-based split-aptamers can be realized (FIG. 13). This observation showed that the FIT-aptamer approach can be used for detecting analytes that do not have well-characterized aptamer binding sites or do not support two-epitope binding.

In summary, a new class of signaling aptamers based on forced intercalation is described and shown herein. It was shown that highly sensitive FIT-aptamers can be designed for the most common aptamer-target binding modes, exemplifying that FIT-aptamers constitute a new paradigm for simple, versatile, and reliable detection. FIT-aptamers provide key advantages over state-of-the-art fluorescence-based signaling aptamers. For example, they are kinetically superior to strategies that require partial blocking of the aptamer site and show higher signal-to-background ratio than traditional FRET-based techniques. Furthermore, a FIT-based strategy enables the probing of local target-induced conformational changes, a powerful capability that is not possible with conventional fluorophore/quencher or FRET-based methodologies. The ability to detect thrombin in human serum bodes well for extending the FIT-aptamer strategy to a wide variety of biological detection scenarios.

### Example 2

### Materials and Methods

### Oligonucleotide Design, Synthesis, Purification, and Characterization

Oligonucleotide design. Table 1 lists all the oligonucleotide sequences used in this study. The dye, D, is represented throughout the disclosure by “D”.

TABLE 1
Oligonucleotide sequences described herein.

| | SEQUENCE | SEQ | |
|---|---|---|---|
| ABBREVIATION | (FROM 5’ END TO 3’ END) | ID NO: | PURPOSE |
| HGA1 | TCATG TTTGT TTG TTG GCC CCC | 1 | Aptamer For Hg²⁺ |
| | CTT CTT TDT TA | | |
| HGA1COMP | CAA ACA AAC ATG A | 2 | Short Complement To Hga1 |
| HGA2 | TTT D TTT TTT T TTT | 3 | Simpler/Shorter Aptamer |
| | | | For Hg²⁺ |
| HGA2-F | TTT T TTT TTT T TTT-FLUORESCEIN | 4 | Strands For Fret |
| HGA2-T | TAMRA dT-TTT T TTT TTT T TTT | 5 | Experiments With Hg²⁺ |
| HGA2-FT | TAMRA dT-TTT T TTT TTT T TTT- | 6 | |
| | FLUORESCEIN | | |
| AGA | CCC D CCC AAAA CCC T CCC | 7 | Aptamer For Ag⁺ |
| I-MD | CCCC TAA CDCC TAA CCCC TAA | 8 | Aptamer For H⁺ |
| | CCCC | | |
| THR1D | GGT TGG TGT GGT TGG (SPACER18)₅ | 9 | Two Aptamers That Bind |
| | CGC D TCT | | Two Distinct Epitopes On |
| THR2 | AGA T GCG (SPACER18)₅ AGT CCG | 10 | Thrombin |
| | TGG TAG GGC AGG TTG GGG TGA CT | | |
| THR1-F | GGT TGG TGT GGT TGG (SPACER18)₅ | 11 | Strands For Fret |
| | CGC A TCT-FLUORESCEIN | | Experiments For Two |
| THR2-T | TAMRA dT-AGA T GCG (SPACER18)₅ | 12 | Epitope Thrombin Binding |
| | AGT CCG TGG TAG GGC AGG TTG | | |
| | GGG TGA CT | | |
| THR1-C | GGT TGG TGT GGT TGG (SPACER18)₅ | 13 | Control Sequences For Fret |
| | CGC A TCT | | Experiments For Two |
| THR2-C | T AGA T GCG (SPACER18)₅ AGT CCG | 14 | Epitope Thrombin Binding |
| | TGG TAG GGC AGG TTG GGG TGA CT | | |
| SPLIT-THRA | TGG TTG G TTTTTT (SP18)5 C GCD | 15 | Split Aptamers For |
| | TCT | | Thrombin |
| SPLIT-THRB | AGA TGC G (SP18)5 AAAAAA GGT | 16 | |
| | TGG TG | | |
| 5’-TDT-3’ | ATA TTC TGA A TDT CA TCC TGC | 17 | DNA Sequences Used To |
| 5’-TDA-3’ | ATA TTC TGA A TDA CA TCC TGC | 18 | Demonstrate That D Can |
| 5’-ADA-3’ | ATA TTC TGA A ADA CA TCC TGC | 19 | Report The Formation Of T- |
| 3’-TGT-5’* | GCA GGA TG TGT T TCA GAA TAT | 20 | Hg²⁺-T Base Pairing Within |
| 3’-TGA-5’* | GCA GGA TG AGT T TCA GAA TAT | 21 | A Preformed |
| 3’-AGA-5’* | GCA GGA TG AGA T TCA GAA TAT | 22 | Oligonucleotide Duplex |

*This sequence reads “...AGT...” from 5’-3’. However, it is named as 3 l’-TGA-5’ as it reads “...TGA...” when written from 3’-5’.

Ligand-induced intramolecular conformational changes. A DNA sequence (5′-TCATG TTTGT TTG TTG GCC CCC CTT CTT TCT TA-3′ (SEQ ID NO: 23)) was first selected that is known to bind to Hg²⁺ via intramolecular conformational changes.⁴⁵ The underlined cytosine at the 4th position from the 3′ end was replaced with D to obtain a FIT-aptamer (HgA1). It was anticipated that in the presence of Hg²⁺, T-Hg²⁺-T base pairing would occur, turning on the fluorescence of D (FIG. 3).

It is noted that the sequence used has been previously shown to detect Hg²⁺ as a structure-switching signaling aptamer that incorporates a fluorophore-quencher pair. This strategy necessitated that part of the aptamer be blocked by a short complementary strand.⁴⁵ To study the impact of the short complement on the response time of the probe, a 13-mer complement was synthesized (HgA1comp).

The FIT-aptamer HgA2 was next synthesized, consisting of a simple string of 14 thymine bases with the 4th base from the 5′ end replaced with D (FIG. 4A). To study how FIT-aptamers compare with FRET-based platforms, three additional T14 sequences were synthesized: the first sequence (HgA2-F) was labeled with fluorescein at the 3′ end, the second sequence (HgA2-T) was labeled with TAMRA dT at the 5′ end, and the third sequence (HgA2-FT) was labeled with both fluorescein and TAMRA at the 3′ and 5′ ends, respectively. Fluorescein and TAMRA are a well-known FRET pair. Hg²⁺-mediated thymine coordination in HgA2-FT is expected to bring the FRET pair into close proximity, resulting in a FRET signal (FIG. 5). HgA2-F and HgA2-T were synthesized as control sequences, to study the influence of Hg²⁺ on the FRET signal in the absence of one of the dyes.

As a simple probe for Ag⁺, a cytosine-rich strand was designed (AgA, FIG. 6), while a modified sequence for the human telomeric i-motif was used to detect pH changes (I-mD, FIG. 9A).

Aptamer-based sandwich assay. Thrombin was chosen as a model target for studying two epitope aptamer binding. Aptamers known to associate with thrombin at two distinct sites were used in this study.⁴⁶ To generate FIT-aptamers, five spacer18 moieties and 7-base complementary sequences were appended to the 3′ end of the first aptamer (THR1 D) and 5′ end of the second aptamer (THR2), respectively. The 4th base from the 3′ end of THR1D was replaced with D (FIG. 10A).

Split-aptamers. Thrombin was chosen as a model target for studying the feasibility of designing FIT-based split-aptamers. A known aptamer for thrombin was fragmented into two parts.⁴⁷ This aptamer is known to form a G-quadruplex and then bind to thrombin. To aid in G-quadruplex formation, complementary bases were appended to the 5′ and 3′ ends of the two aptamers, respectively. So that the designed FIT-aptamers reports target presence, a similar design strategy as used for two epitope binding was employed: five spacer18 moieties and 7-base complementary sequences were appended to the 3′ end of the first aptamer (Split-THRa) and 5′ end of the second aptamer (Split-THRb), respectively. The 4th base from the 3′ end of Split-THRa was replaced with D (FIG. 13).

Intraduplex metallo-base pair formation. Three 21-base DNA sequences were synthesized that differed only in terms of the bases at positions 11, 12, and 13 from the 5′ end. The sequences consisted of TDT, TDA, and ADA regions and these sequences were correspondingly termed as 5′-TDT-3′, 5′-TDA-3′, and 5′-ADA-3′, respectively. Three strands that are complementary to these sequences were also synthesized: 3′-AGA-5′, 3′-AGT-5′, and 3′-TGT-5′. The dye-labeled strands were added to the unlabeled strands in different combinations which resulted in the formation of duplexes with varying numbers of T-T mismatches adjacent to D (Table 2).

TABLE 2
Number of T-T mismatches in DNA duplexes

| | 5′-TDT-3′ | 5′-TDA-3′ | 5′-ADA-3′ |
|---|---|---|---|
| 3′-AGA-5′ | 0 | 0 | 0 |
| 3′-AGT-5′ | 1 | 0 | 0 |
| 3′-TGT-5′ | 2 | 1 | 0 |

Synthesis, purification, and characterization. DNA synthesis reagents were purchased from Glen Research. Oligonucleotides were synthesized either on a MerMadel 2 (MM12, BioAutomation Inc., Plano, Tex., USA) instrument using universal controlled pore glass (CPG) beads at a 5 μmol scale or an ABI 394 using CPG beads at a 1 μmol scale. After synthesis, the CPG-bound oligonucleotides were cleaved from the CPG beads and deprotected for 4 h at 55° C. using 2 mL of 30% ammonium hydroxide. The ammonia was then evaporated off using an Organomation® Multivap® Nitrogen Evaporator. The volume of the remaining solution was adjusted to 2 mL using nanopure water. The solution was then filtered through a 0.2 μM syringe filter to remove the CPG beads. Subsequent purification was performed using reverse phase high pressure liquid chromatography (RP-HPLC, Varian ProStar 210, Agilent Technologies Inc., Palo Alto, Calif., USA) employing a C18 column and a gradient of 0 to 75% B over 45 min (A=triethylammonium acetate buffer, B=acetonitrile). Collected fractions were then lyophilized overnight and subsequently detritylated with 2 mL of 20% acetic acid for 1 hour. The cleaved DMTr was extracted using ethyl acetate. The remaining acidic oligonucleotide solution was lyophilized overnight again, and resuspended in nanopore water. The fraction containing the product was identified using matrix-assisted laser desorption ionization time-of-flight mass spectrometry (MALDI-TOF MS). The concentration of product was determined via UV-VIS spectroscopy using the extinction coefficient at 260 nm obtained from the IDT Oligo Analyzer Tool.

### Synthesis and Characterization of Quinoline Blue (D)

General methods. All chemicals, reagents, and solvents were purchased as reagent grade from Sigma-Aldrich, Acros, or Alfa Aesar and used as received unless otherwise stated. When specified, solvents were degassed under a stream of argon before use. All glassware and stir bars were oven-dried at 180° C. Flash chromatography was performed using SiO₂ 60 (230-400 mesh ASTM, 0.040-0.063 mm; Fluka). Deuterated solvents were purchased from Cambridge Isotope Laboratories and used as received. ¹H and ¹³C NMR spectra were recorded on a Bruker Avance 400 MHz NMR spectrometer at 298 K, and chemical shifts (δ) are given in parts per million. ¹H NMR spectra were referenced to residual proton resonances in the deuterated solvents (dimethylsulfoxide-d₆=δ 2.50), while absolute referencing was applied for heteronuclear NMR spectra (Ξ_{C}=25.145020). Electrospray ionization mass spectrometry (ESI-MS) was recorded on a Micromas Quatro II triple-quadrapole mass spectrometer in positive ion mode. N-methyl-4-chlorquinolinium iodide and N-carboxylmethyl-4-methquinolinium bromide were synthesized following literature procedures.^{48,49}

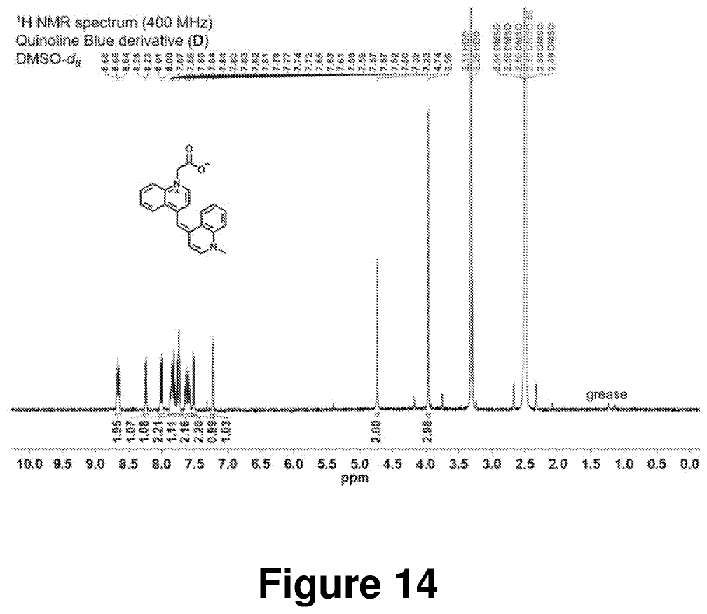

Synthesis of quinoline blue derivative. D was synthesized as described previously with slight modifications.⁵⁰ N-methyl-4-chlorquinolinium iodide (0.2636 g, 0.863 mmol, 1 equiv) and N-carboxylmethyl-4-methquinolinium bromide (0.292 g, 1.035 mmol, 1.2 equiv) were suspended in 2.2 mL of dichloromethane (CH₂Cl₂). To the brown suspension was added trimethylamine (0.4190 mL, 3.02 mmol, 3.5 equiv), and the resulting solution turned dark blue immediately. The mixture was stirred at room temperature in the dark overnight. The mixture was then dried and purified via flash chromatography (CH₂Cl₂:MeOH=8:2). The product fractions were combined and dried in vacuo to obtain a bluish-purple powder (0.2083 g, 0.608 mmol, isolated yield=71%). The product is stable when stored dried and shielded from light, but a solution of D would decolorize and degrade when exposed to light for an extensive period of time. ¹H NMR (400 MHz, DMSO-d₆) δ 8.71-8.62 (m, 2H), 8.24 (d, J=7.1 Hz, 1H), 8.00 (d, J=7.4 Hz, 1H), 7.90-7.82 (m, 2H), 7.82-7.78 (m, 1H), 7.78-7.70 (m, 2H), 7.67-7.54 (m, 2H), 7.51 (d, J=7.5 Hz, 1H), 7.23 (s, 1H), 4.74 (s, 2H), 3.96 (s, 3H). ¹³C NMR (101 MHz, DMSO-d₆) δ 165.89, 149.40, 147.26, 144.21, 142.43, 138.62, 138.58, 132.12, 132.09, 125.89, 125.54, 125.44, 125.35, 125.27, 124.62, 118.39, 117.15, 109.32, 106.83, 95.72, 59.35, 41.09. ESI-MS m/z Calcd for [M+H]⁺: 343.14 m/z. Found: 343.14 m/z. Calcd for [M+Na]⁺: 365.13 m/z. Found: 365.10 m/z. See FIG. 14.

Coupling of D to oligonucleotide probes. D-carboxylate was coupled to oligonucleotide probes containing an amino-modifier (N-trifluoroacetyl serinol phosphoramidite) using a previously reported protocol with modifications (FIG. 2).⁵⁰ In a typical experiment, D-carboxylate (5 μmol) and pyridinium para-toluene sulfonate (5 μmol) were added to dimethylformamide (250 μL) and vortexed to dissolve the components. Thereafter, N-hydroxysuccinimide (25 μmol) and 1-Ethyl-3-(3-dimethylaminopropyl) carbodiimide (50 μmol) were added to the solution, and the mixture was incubated at 30° C. for 10 min. This activated dye solution was added to 100 nmol of oligonucleotides (dissolved in 250 μL of 0.1 M NaHCO3) and allowed to incubate for 2 hours.

The dye-oligonucleotide solution was added to water (15 mL) and filtered (3 kDa Amicon Ultra-15 centrifugal filter units, Millipore Sigma). The filtrate was discarded and the solution remaining in the filter was purified using a NAP™-10 (GE Healthcare) column to remove any unreacted dye. The dye-conjugated oligonucleotide was separated from unreacted oligonucleotides using RP-HPLC with a C18 column and a gradient of 0 to 75% B over 45 min (A=triethylammonium acetate buffer, B=acetonitrile). The product containing fraction was analyzed using MALDI-TOF and quantified using UV-Vis spectroscopy as before.

### Fluorescence Experiments

All measurements were performed in triplicate unless otherwise mentioned.

### 1. Aptamer-Hg²⁺ Binding Studies

### HgA1

Calibration curve. A calibration curve measuring the response of probe HgA1 to target in MOPS buffer (10 mM MOPS, 100 mM NaNO3, pH 7.2) was constructed using a BioTek Synergy H4 Hybrid Reader. 50 nM HgA1 probe was challenged with different amounts (0, 25, 50, 75, 100, 150, 200, 250, and 300 nM) of HgCl₂ in different wells of a 96 well plate. The fluorescence reading was taken at 20° C.

Kinetics. 50 nM HgA1 probe (with and without 100 nM of HgA1comp) was challenged with 250 nM HgCl2 in MOPS buffer (10 mM MOPS, 100 mM NaNO3, pH 7.2) at 20° C. The fluorescence was read using an ISS PC1. Fluorescence of just the probe was read before the addition of Hg²⁺, and then monitored until the signal saturated after HgCl₂ addition.

### HgA2

Effect of pH on binding. 250 nM HgA2 probe was challenged with 75 nM HgCl₂ in varying pH Britton-Robinson (BR) buffer (pH 6, 7, 8, 9, 10, and 11) and fluorescence was read using an ISS PC1 at 20° C. Fluorescence of the probe was read both before and after the addition of Hg²⁺.

Effect of ionic strength. 250 nM HgA2 probe was challenged with 75 nM HgCl₂ in pH 10 BR buffer at different ionic strengths (0, 100, and 500 mM NaCl) and fluorescence was read using an ISS PC1. Fluorescence of just the probe was read before the addition of HgCl₂. Fluorescence readings were taken at 20° C.

Calibration curve. All fluorescence readings were taken on an ISS PC1 at 20° C. In a representative experiment, a fluorescence reading was first taken of pH 10 BR buffer. Then, 50 nM HgA2 probe was added and another fluorescence reading was taken. Lastly, x nM HgCl₂ (where x is 10, 25, 50, 75, 100, 150, 200, or 250) was added and a fluorescence reading was taken. The cuvette was then washed, and this procedure was repeated for the next value of x. A corresponding spectrum was taken for each of these points by excitation at 560 nm and collection of emission from 590 nm to 750 nm.

Selectivity. 50 nM HgA2 probe in pH 10 BR buffer was challenged with 500 nM of varying metals (Hg²⁺, Ag⁺, Pb²⁺, NH₄⁺, K⁺, Fe³⁺, Fe²⁺, Mn²⁺, Mg²⁺, Cu²⁺, Ca²⁺, Co²⁺, Ni²⁺, Zn²⁺, Cd²⁺, and Ba²⁺) and fluorescence was measured using a BioTek Synergy H4 Hybrid Reader. The fluorescence readings were taken at 20° C.

FRET experiments. All experiments were done in pH 10 BR buffer and fluorescence was collected using a BioTek Synergy H4 Hybrid Reader at 20° C. Excitation was performed at 480 nm and emission was collected at 520, 580, and 700 nm. 50 nM of HgA2-F, 50 nM of HgA2-T, or 50 nM of HgA2-FT probe were challenged with 500 nM HgCl₂.

### 2. Aptamer-Ag⁺ Binding Studies

Calibration curve. All fluorescence readings were taken on an ISS PC1 at 20° C. In all experiments, 50 nM AgA probe was added to MOPS buffer (10 mM MOPS, 100 mM NaNO₃, pH 7.2). In a representative experiment, a fluorescence reading was first taken of the MOPS buffer. Then, 50 nM AgA probe was added and another fluorescence reading was taken. Lastly, x nM Ag⁺ (where x is 10, 25, 50, 100, 250, 500, 750, or 1000) was added and a fluorescence reading was taken. The cuvette was then washed, and this procedure was repeated for the next value of x. A corresponding spectrum was taken for each of these points by excitation at 560 nm and collection of emission from 590 nm to 750 nm.

Selectivity. 50 nM AgA probe in MOPS buffer (10 mM MOPS, 100 mM NaNO3, pH 7.2) was challenged with 500 nM of varying metals (Ag⁺, Hg²⁺, Pb²⁺, NH₄⁺, K⁺, Fe³⁺, Fe²⁺, Mn²⁺, Mg²⁺, Cu²⁺, Ca²⁺, Co²⁺, Ni²⁺, Zn²⁺, Cd²⁺, Ba²⁺) and fluorescence was measured using a BioTek Synergy H4 Hybrid Reader. The fluorescence reading was taken at 20° C.

3. Studies with i-Motif.

All fluorescence readings were taken on an ISS PC1 at 25° C. 50 nM I-mD was added to pH x McIlvaine buffer (where x is 5.4, 5.6, 5.8, 6, 6.2, 6.4, 6.6, 6.8, 7.0, or 7.6). In a typical experiment, a fluorescence reading of pH x McIlvaine buffer was first taken. Then, 50 nM I-mD was added and a fluorescence reading was taken. The cuvette was washed, and this procedure was repeated for the remaining values of x. The procedure was repeated in full using the HgA2 probe as a control. Corresponding spectra were collected for the I-mD probe at pH 5.4, 5.6, 5.8, 6.0, 6.2, 6.6, 7.0, and 7.6 by exciting at 560 nm and collecting emission from 590 nm to 750 nm.

### 4. Two-Aptamer Binding to Thrombin

Effect of temperature on binding. All fluorescence readings were taken on an ISS PC1. In a typical experiment, a fluorescence reading was first taken of 1× Tris-buffered saline (20 mM Tris, 0.9% NaCl, pH 7.4). Then, 50 nM THR1D and 50 nM THR2 were added to 1× Tris-buffered saline and a fluorescence reading was taken after 3 minutes. 100 nM thrombin was then added, and a fluorescence reading was taken after 3 minutes. In this experiment, this procedure was done at 4 different temperatures (5, 10, 15, and 20° C.) to determine the temperature of maximum fluorescence enhancement.

Calibration curve. All fluorescence readings were taken on an ISS PC1 at 10° C. In a typical experiment, a fluorescence reading was first taken of 1× Tris-buffered saline (20 mM Tris, 0.9% NaCl, pH 7.4). Then, 50 nM THR1D and 50 nM THR2 were added to 1× Tris-buffered saline and a fluorescence reading was taken after 3 minutes. x nM thrombin (where x is 2.5, 5, 10, 17.5, 25, 50, or 100) was then added, and a fluorescence reading was taken after 3 min. The cuvette was then washed, and the procedure was repeated for the remaining values of x. A corresponding spectrum was taken for each of these points by excitation at 560 nm and collection of emission from 590 nm to 750 nm.

Selectivity. All fluorescence readings were taken on an ISS PC1 at 10° C. In a typical experiment, a fluorescence reading was first taken of 1× Tris-buffered saline (20 mM Tris, 0.9% NaCl, pH 7.4). Then, 50 nM THR1D and 50 nM THR2 were added to 1× Tris-buffered saline and a fluorescence reading was taken after 3 minutes. 25 nM protein (where protein is thrombin, proteinase K, immunoglobulin G, hemoglobin, elastase, or single-strand DNA-binding protein) was then added, and a fluorescence reading was taken after 3 minutes. The cuvette was then washed, and the procedure was repeated for the remaining proteins.

FRET experiments. All fluorescence readings were taken on an ISS PC1 at 10° C. in 1× Tris-buffered saline (20 mM Tris, 0.9% NaCl, pH 7.4). Six different spectra were collected by exciting at 440 nm and collecting emission from 470 nm to 700 nm: (i) 1× Tris-buffered saline alone (ii) 50 nM THR1-F in 1× Tris-buffered saline, (iii) 50 nM probe THR1-F and 50 nM probe THR2-T in 1× Tris-buffered saline (after 3 min incubation), and (iv-vi) 50 nM THR1-F, 50 nM probe THR2-T, and x nM thrombin (where x was 5, 10, or 50) in 1× Tris-buffered saline (after 3 minute incubation).

### 5. Split-Aptamer

Calibration curve. All fluorescence readings were taken on an ISS PC1 at 10° C. In a typical experiment, a fluorescence reading was first taken of 1× Tris-buffered saline with 100 mM KCl (20 mM Tris, 0.9% NaCl, 100 mM KCl, pH 7.4). Then, 50 nM Split-THRa and 50 nM Split-THRb were added to the buffer and a fluorescence reading was taken after 3 minutes. x nM thrombin (where x is 25, 50, 100, 200, or 300 nM) was then added, and a fluorescence reading was taken after 3 minutes. The cuvette was then washed, and the procedure was repeated for the remaining values of x. A corresponding spectrum was taken for each of these points by excitation at 560 nm and collection of emission from 590 nm to 750 nm.

Selectivity. All fluorescence readings were taken on an ISS PC1 at 10° C. In a typical experiment, a fluorescence reading was first taken of 1× Tris-buffered saline with 100 mM KCl (20 mM Tris, 0.9% NaCl, 100 mM KCl, pH 7.4). Then, 50 nM Split-THRa and 50 nM Split-THRb were added to buffer and a fluorescence reading was taken after 3 minutes. 25 nM protein (where protein is thrombin, proteinase K, immunoglobulin G, hemoglobin, elastase, or single-strand DNA-binding protein) was then added, and a fluorescence reading was taken after 3 minutes. The cuvette was then washed, and the procedure was repeated for the remaining proteins.

### 6. Intraduplex Metallo-Base Pair

All experiments were done in 1×PBS (11.9 mM Phosphates, 137 mM NaCl, 2.7 mM KCl, pH 7.4) and fluorescence measurements were performed using a BioTek Synergy H4 Hybrid Reader at 20° C. In a typical experiment, 50 nM of a dye-labeled sequence was mixed with 50 nM of a complementary unlabeled sequence. The fluorescence was recorded in the absence and presence of HgCl₂. This procedure was carried out for all dye labeled sequence/complement combinations (i.e. TDT/TGT, TDT/TGA, TDT/AGA, TDA/TGT, TDA/TGA, TDA/AGA, ADA/TGT, ADA/TGA, and ADA/AGA). As a control, fluorescence readings were taken individually of just 50 nM of the dye-labeled sequences in the absence and presence of 500 nM Hg²⁺.

### 7. Detection of Thrombin in Serum

All fluorescence readings were taken on an ISS PC1 at 20° C. One-part whole serum was diluted with 4-parts 1.25× Tris buffered saline (30 mM Tris, 1.13% NaCl, pH 7.4) to yield a 4:1 Tris buffered saline:serum buffer. This buffer was then filtered through a 0.2 μm filter. In a typical experiment, 50 nM THR1D and 50 nM THR2 were added to the serum/buffer mixture and a fluorescence reading was taken after 3 minutes. x nM thrombin (where x is 2.5, 5, 10, or 50) was then added, and a fluorescence reading was taken after 2 minutes. The cuvette was then washed, and the procedure was repeated for the remaining values of x.

### CD Studies

Circular dichroism (CD) studies were performed using a Jasco J-1700 circular dichroism spectrometer. All samples were placed in a low-volume quartz cuvette with a 1 cm path length and the spectra were collected between 230-330 nm. HgA1, HgA2, and AgA were dissolved in 1×MOPS (pH 7.2, with 0.1 M NaNO₃), BR buffer (pH 10), and 1×MOPS (pH 7.2, 0.1 M NaNO₃), respectively, at a concentration of 2.5 μM. The spectra were recorded in the absence or presence of 10 equivalent target (Hg²⁺ or Ag⁺). In the case of HgA1, additional spectra were recorded in the presence of 5 μM HgA1comp. Similarly, 1.25 μM of the i-motif was dissolved in McIlvaine buffer at different pH (7.0, 6.4, 6.2, 6.0, 5.8, 5.6, 5.4, or 5.2). The CD value at 290 nm, characteristic of i-motif formation, was monitored as a function of pH. In all cases, the spectra were corrected for the contribution from the buffers.

### Data Analysis

The values and error bars in all in graphs and charts represent the average and standard deviation, respectively, of 3 independent readings. The fluorescence enhancement was calculated according to Equation 1:

$$E . F . = \frac{{I}_{f}}{{I}_{0}} \qquad eq ( 1 )$$

I₀ represents the initial fluorescence of the probe alone while I_{f} represents the fluorescence observed upon addition of analyte.

The calibration curves for HgA1, HgA2, and AgA were fit to a Hill-equation (Equation 2) to estimate the dissociation constant (K).

$$y = {y}_{0} + \frac{{y}_{m} - {y}_{0}}{1 + {( \frac{K}{x} )}^{n}} \qquad eq ( 2 )$$

x represents the concentration of the analytes added and y represents the corresponding fluorescence enhancement. y₀ and yₘ represent the initial and maximum fluorescence enhancements and n represents the Hill-coefficient. n>1 is observed in all cases and is indicative of cooperative binding, consistent with previous reports. It should be noted that in this study, K is reflective of the concentration of the analyte that causes half of the maximum fluorescence enhancement possible.

For I-mD, the data was fit to a sigmoidal curve (Equation 3):

$$y = {y}_{0} + \frac{{y}_{m} - {y}_{0}}{1 + 1 {0}^{( a - x ) * b}} \qquad eq ( 3 )$$

Similar to Equation 2, x represents pH and y represents the corresponding fluorescence enhancement. y₀ and yₘ represent the initial and maximum fluorescence enhancements. a represents the pH at which half of the maximum fluorescence enhancement possible is observed. b is a fitting parameter corresponding to the slope of the transition region.

For all analytes, the limit of detection (LOD) was determined by the 3σ/m method, where σ denotes the standard deviation of the response and m denotes the initial slope of the calibration curve.

### Intramolecular Conformational Changes

Studies with HgA1 and HgA1comp. As noted above, a DNA sequence (5′-TCATG TTTGT TTG TTG GCC CCC CTT CTT TCT TA-3′(SEQ ID NO: 23)) was used that is known to bind to Hg²⁺ via intramolecular conformational changes.⁴⁵ The underlined cytosine at the 4th position from the 3′ end was replaced with D to obtain a FIT-aptamer (HgA1). By placing D between two thymines, it was hypothesized that mercury induced coordination of thymines would result in the forced intercalation of D between the metallo-bps, therefore turning on fluorescence (FIG. 3A).

To assess this hypothesis, the fluorescence response of 50 nM HgA1 was measured in the absence and presence of varying concentrations of mercury in MOPS buffer (10 mM MOPS, 100 mM NaNO₃, pH 7.2). A maximal 8-fold enhancement is observed at 250 nM and a LOD of 22.95 nM is found (FIG. 3B).

In previous literature, HgA1 has been used as part of a structure-switching signaling aptamer for detecting mercury. In the previous system, the 5′ end of 5′-TCATG TTTGT TTG TTG GCC CCC CTT CTT TCT TA-3′ (SEQ ID NO: 23) (i.e. HgA1 without the underlined cytosine being replaced by D) was labeled with a fluorophore and hybridized with a short 3′ quencher labeled complement. In the fluorescence “off state” of the probe, the two sequences were hybridized, thus placing the fluorophore and quencher in close proximity. Mercury induced coordination of thymines in the fluorophore-labeled strand resulted in its folding and subsequent displacement of the quencher-labeled strand, yielding fluorescence turn-on. However, partial blocking of the aptamer site has been shown to slow probe response.⁴⁵

To simulate a structure switching aptamer in order to study how a short complement partially blocking the aptamer impacts probe response time, a 13-mer complement to HgA1 (HgA1comp, FIG. 3C) was synthesized. The change in fluorescence was evaluated over time after addition of mercury to HgA1 only versus HgA1 partially blocked through prehybridization to HgA1comp. The results indicated that partial blocking of the aptamer site slows probe response time by approximately 5-fold (FIG. 3D). Because a FIT-based system does not require partial blocking of the aptamer site (in contrast to structure-switching signaling aptamers), its kinetic response is superior to structure-switching signaling systems.

Studies with HgA2

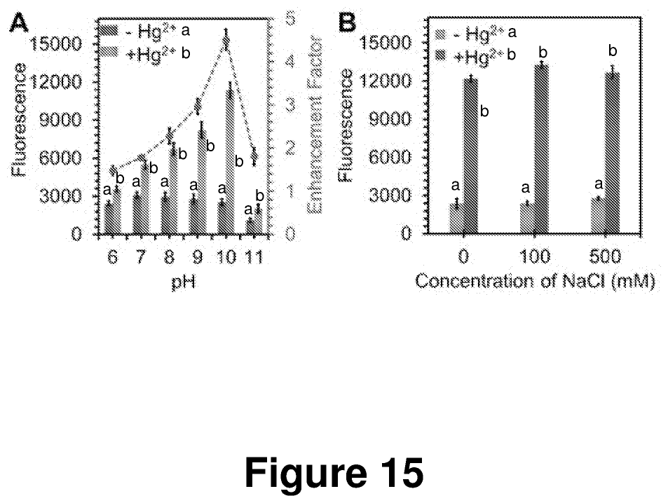

Effect of pH and ionic strength on fluorescence enhancement. The pH at which addition of Hg²⁺ results in maximum fluorescence enhancement was determined first. The fluorescence of 250 nM HgA2 dissolved in a buffered solution (pH 6-11) was monitored in the absence and presence of 75 nM Hg²⁺. In order to keep the buffer composition the same across the different pH studied, we used BR buffer, considered to be a “universal buffer”, which was titrated to the desired pH using NaOH. The results showed that at pH 10, maximum fluorescence enhancement is observed (FIG. 15A). It is believed this is because the pKa of thymine is 9.5.⁵¹ Hg²⁺ competes with H⁺, and therefore, at pH 10, the binding of Hg²⁺ is favored over H⁺.

It was noted that in a realistic sample, the ionic strength can vary significantly (0-600 mM) depending on the source of the sample (e.g. water from lakes, seas, ponds, etc.). Therefore, the effect of ionic strength on fluorescence enhancement was investigated next. As before, 75 nM Hg²⁺ was added to 250 nM HgA2 in BR buffer of pH 10. The ionic strength of the buffer was varied by adding different concentrations of NaCl. The results (FIG. 15B) showed that the fluorescence enhancement changes negligibly over an ionic strength of 0-500 mM. Therefore, further experiments (FIG. 4) were performed in BR buffer of pH 10 without any added NaCl.

FRET experiments. Using a T₁₄ sequence (as used in HgA2) it is possible to imagine a probe that incorporates a FRET pair at its two ends such that Hg²⁺ binding brings them into close proximity resulting in a FRET signal (FIG. 5A). To study how the FIT-aptamer compared with a traditional FRET-based probe, a T₁₄ strand labeled with fluorescein at the 3′ end and TAMRA at the 5′ end (HgA2-FT) was synthesized. As controls, two additional sequences were synthesized: HgA2-F which consisted of a T₁₄ sequence with only fluorescein at the 3′ end and HgA2-T, comprised of a T₁₄ sequence with only TAMRA at the 5′ end.

500 nM Hg²⁺ was added to 50 nM of HgA2-FT, HgA2-F, and HgA2-T. The fluorescence of the donor (fluorescein) channel was monitored in the absence and presence of Hg²⁺ (FIG. 5B) and used to calculate the FRET efficiency (E) via Equation 4:

$$E = ( 1 - \frac{{F}_{donor \to acceptor}}{{F}_{acceptor}} ) * 100 \% \qquad eq ( 4 )$$

F_{(donor→acceptor)} is the fluorescence of the donor in the presence of the acceptor (i.e. donor channel fluorescence for HgA2-FT) and F_{acceptor} is the fluorescence of the acceptor alone (i.e. donor channel fluorescence of HgA2-F). The signal to noise ratio was calculated by taking the ratio of the FRET efficiency in the presence of Hg²⁺ to the FRET efficiency in its absence.

A relatively high FRET efficiency (approximately 71%) was observed even in the absence of Hg²⁺. This result was expected given the short sequence length as well as the small persistence length of single-stranded DNA which results in the fluorophores being well within the Forster radius.⁵² Addition of 250 nM Hg²⁺ further decreases the distance between the fluorophores, increasing the FRET efficiency to approximately 93%. Therefore, the signal to background ratio is only 1.32, in sharp contrast to the 20-fold fluorescence enhancement observed using the FIT-aptamer (FIG. 4B). These results showed that FIT-aptamers enable the use of shorter single-stranded probes with superior signal to background ratios compared to FRET.

Studies with AgA

To study whether the FIT-based strategy can detect Ag⁺, a DNA sequence was designed that places D between two cytosines such that the coordination of cytosines by silver results in the forced intercalation of D between the metallo-bps (FIG. 6A). The fluorescence of 50 nM AgA was measured in the absence and presence of varying amounts of Ag in MOPS buffer (10 mM MOPS, 100 mM NaNO₃, pH 7.2). A nearly 5-fold maximal fluorescence enhancement was observed in the presence of 500 nM Ag⁺ (FIG. 6B) and find a limit of detection of 17.27 nM, below the level that the EPA deems as unsafe in drinking water.⁵³ To ensure that AgA is also selective, 50 nM of the probe was challenged with 500 nM of varying metals (FIG. 6C). These results indicated selective turn-on in the presence of silver.

### CD Experiments

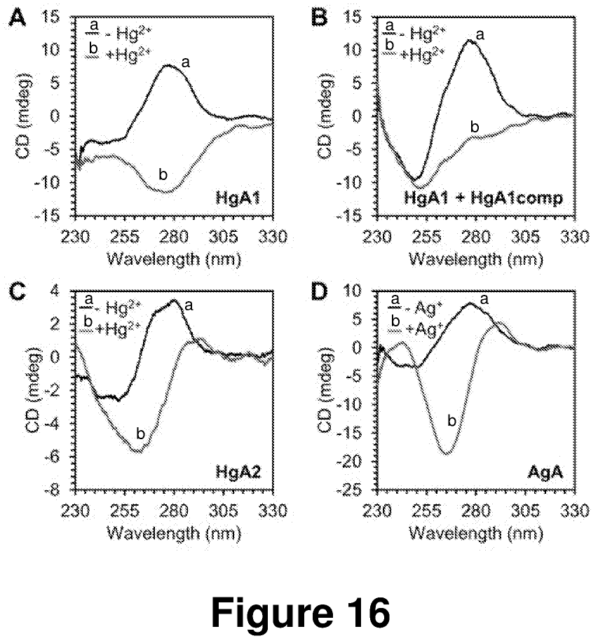

CD experiments were performed on 2.5 μM of the probes (HgA1, HgA1+2 eq. HgA1comp, HgA2, and AgA) in the presence and absence of 25 μM target (Hg²⁺ or Ag⁺). A dramatic change was observed in the CD spectra of the aptamers in the presence of their targets, confirming that the probes undergo conformational changes upon target binding (FIG. 16).

### Two Epitope Binding

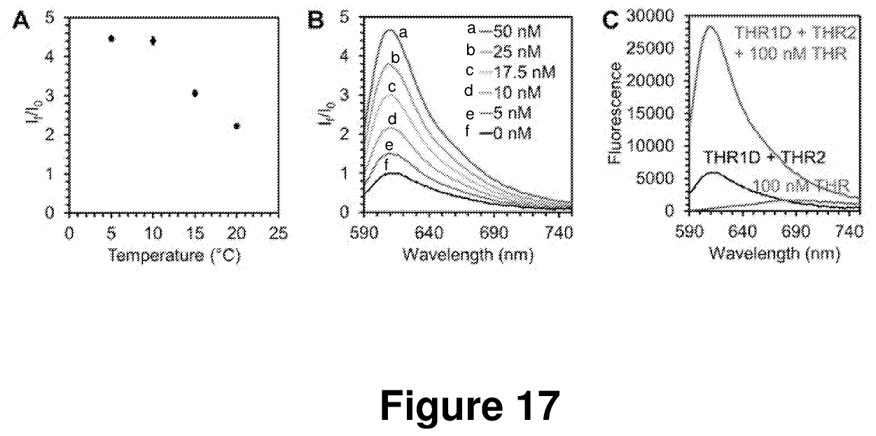

Effect of temperature on fluorescence enhancement. Given that both DNA hybridization and aptamer-target binding are temperature dependent, work was begun by finding the temperature at which maximum fluorescence enhancement is observed upon addition of thrombin. The fluorescence of 50 nM THR1D and 50 nM THR2 in 1× Tris-buffered saline (20 mM Tris, 0.9% NaCl, pH 7.4) was measured in the absence and presence of 100 nM thrombin at 4 different temperatures (5° C., 10° C., 15° C., or 20° C.). Maximal fluorescence enhancement was found to occur at 5° C. and 10° C. (FIG. 17A). Subsequent experiments were performed at 10° C. to facilitate binding kinetics. FIG. 10B in the main text and FIG. 17B show the fluorescence enhancements obtained upon addition of varying amounts of thrombin to the FIT-aptamers.

Fluorescence of thrombin. To ensure that thrombin itself does not contribute to fluorescence signal at the monitored wavelength (610 nm), the fluorescence of 1× Tris-buffered saline was monitored in the presence of 100 nM thrombin. The results (FIG. 17C) show that the fluorescence of thrombin has a negligible contribution to the fluorescence signal at 610 nm.

FRET experiments. To compare the FIT-based detection method with a conventional FRET methodology in the context of two epitope binding to thrombin, sequences THR1-F with a fluorescein at the 3′ end and THR2-T with a TAMRA at the 5′ end were synthesized. The binding of THR1-F and THR2-T to their respective epitopes allows for the complementary regions of the probes to hybridize, bringing the FRET pair into close proximity (FIG. 11A). Excitation and emission spectra of 50 nM THR1-F and 50 nM THR2-T were first acquired to determine optimal excitation/emission wavelengths to use for future experiments (FIG. 11B). Based on these results, the fluorescence of 50 nM THR1-F and 50 nM THR2-T was monitored in the absence and presence of different concentrations of thrombin (10 and 50 nM) using an excitation of 480 nm and collecting emission at 520 nm to calculate the FRET efficiencies (FIG. 11C-D). The results showed a maximal approximately 1.4-fold increase in FRET efficiency at 50 nM thrombin. In comparison, an approximate 5-fold enhancement is observed when the FIT strategy was employed, adding 50 nM thrombin to 50 nM THR1D and 50 nM THR2 (FIG. 11E).

As a control, strands THR1-F and THR2-T were also synthesized without fluorophores (THR1-c and THR2-c, respectively). The fluorescence of 50 nM of each fluorophore labeled probe was monitored in the absence and presence of 50 nM of its complementary control probe (i.e. THR1-F+THR2-c and THR2-T+THR1-c) to ensure that changes in fluorescence intensity were not due to duplexing alone.

Detection of thrombin in human serum. To simulate the detection of an analyte in a realistic scenario, we sought to detect thrombin in human serum. The fluorescence of 50 nM THR1D and 50 nM THR2 was measured in the absence and presence of varying amounts of thrombin in a buffered solution consisting of 4 parts 1.25× Tris-buffered saline and one-part whole serum. The fluorescence enhancement saturates at approximately 2-fold (FIG. 12) and a limit of detection of 1.36 nM is found. Because the serum sample was diluted 5-fold, the limit of detection in whole serum is 6.8 nM, still below the 10 nM critical thrombin concentration in serum associated with blood clot formation.

### Split-Aptamer

Calibration curve. To study the response of the split aptamer system (described herein above and shown in FIG. 13A) to thrombin, a calibration curve was constructed in 1× Tris-buffered saline. The fluorescence of 50 nM Split-THRa and 50 nM Split-THRb was measured in the presence and absence of varying amounts of thrombin. The results showed a maximal approximately 3-fold fluorescence enhancement at 200 nM thrombin and an LOD of 14.76 nM (FIG. 13B).

Selectivity. The fluorescence of 50 nM Split-THRa and 50 nM Split-THRb in 1× Tris-buffered saline was measured in the absence and presence of 25 nM varying proteins (Proteinase K (PK), Immunoglobulin G (IgG), Hemoglobin (Hg), Elastase (Elt), or Single-strand DNA-binding protein (SSDBP)) to confirm that probe turn-on is selective. Probe turn-on was not observed in the presence of these other proteins (FIG. 13C).

### Intraduplex Metallo-Base Pairs

The strands 5′-TDT-3′, 5′-TDA-3′, and 5′-ADA-3′ were added to complementary sequences 3′-AGA-5′, 3′-TGA-5′, and 3′-TGT-5′ in all combinations. Fluorescence readings were taken in the absence and presence of 500 nM Hg²⁺. Control experiments were also performed with the dye-labeled strands alone to ensure addition of Hg²⁺ itself did not increase fluorescence signal. Table 3 qualitatively summarizes the results obtained with explanations and FIG. 8 shows the quantitative fluorescence enhancements (I_{f}/I₀, where I₀ refers to the initial fluorescence of each dye-labeled single strand). In FIG. 7, I₀ refers to the initial fluorescence of a duplexed strand in the absence of Hg²⁺.

TABLE 3
Summary of fluorescence enhancements observed with Hg²⁺ addition to DNA duplexes with varying numbers of internal T-T mismatches.

| | | BUFFER | | 3'-AGA-5' | | 3'-TGA-5' | | 3'-TGT-5' | |
|---|---|---|---|---|---|---|---|---|---|
| | | −Hg²⁺ | +Hg²⁺ | −Hg²⁺ | +Hg²⁺ | −Hg²⁺ | +Hg²⁺ | −Hg²⁺ | +Hg²⁺ |
| | | 1 | 2 | 3 | 4 | 5 | 6 | 7 | 8 |
| 5'-TDT-3' | A | LOW | FLUORES- | FLUORES- | FLUORES- | FLUORES- | FLUORES- | FLUORES- | FLUORES- |
| | | FLUORES- | CENCE | CENCE | CENCE | CENCE | CENCE | CENCE | CENCE |
| | | CENCE | UNCHANGED | INCREASES | UNCHANGED | UNCHANGED | INCREASES | UNCHANGED | INCREASES |
| | | | COMPARED | COMPARED | COMPARED | COMPARED | COMPARED | COMPARED | COMPARED |
| | | | TO A1 | TO A1 DUE | A3 AS NO | TO A1 DUE | TO A5 DUE | TO A1 DUE | TO A7 DUE |
| | | | | TO A-T | T-T | TO T-T | TO T-HG²⁺-T | TO T-T | TO T-HG²⁺-T |
| | | | | BASE | MISMATCHES | MISMATCH | FORMATION | MISMATCHES | FORMATION |
| | | | | PAIRING ON | ARE | ON ONE | ON ONE | ON BOTH | ON BOTH |
| | | | | BOTH SIDES | PRESENT IN | SIDE OF D | SIDE OF D | SIDES OF D | SIDES OF D |
| | | | | OF D | THE DUPLEX | | | | |
| 5'-TDA-3' | B | LOW | FLUORES- | FLUORES- | FLUORES- | FLUORES- | FLUORES- | FLUORES- | FLUORES- |
| | | FLUORES- | CENCE | CENCE | CENCE | CENCE | CENCE | CENCE | CENCE |
| | | CENCE | UNCHANGED | UNCHANGED | UNCHANGED | UNCHANGED | INCREASES | UNCHANGED | INCREASES |
| | | | COMPARED | COMPARED | COMPARED | COMPARED | COMPARED | COMPARED | COMPARED |
| | | | TO B1 | TO B1 DUE | TO B3 AS | TO B1 DUE | TO B5 DUE | TO B1 DUE | TO B7 DUE |
| | | | | TO A-A | NO T-T | TO T-T AND | TO T-HG²⁺-T | TO T-T | TO T-HG²⁺-T |
| | | | | MISMATCH | MISMATCHES | A-A | FORMATION | MISMATCH | FORMATION |
| | | | | ON ONE | ARE | MISMATCHES | ON ONE | ON ONE | ON ONE |
| | | | | SIDE OF D | PRESENT IN | ON THE TWO | SIDE OF D | SIDE OF D | SIDE OF D |
| | | | | | THE DUPLEX | SIDES OF D | | | |
| 5'-ADA-3' | C | LOW | FLUORES- | FLUORES- | FLUORES- | FLUORES- | FLUORES- | FLUORES- | FLUORES- |
| | | FLUORES- | CENCE | CENCE | CENCE | CENCE | CENCE | CENCE | CENCE |
| | | CENCE | UNCHANGED | UNCHANGED | UNCHANGED | UNCHANGED | UNCHANGED | INCREASES | UNCHANGED |
| | | | COMPARED | COMPARED | COMPARED | COMPARED | COMPARED | COMPARED | COMPARED |
| | | | TO C1 | TO C1 DUE | TO C3 AS | TO C1 DUE | TO C5 DUE | TO C1 DUE | TO C7 AS |
| | | | | TO A-A | NO T-T | TO A-A | TO A-A | TO A-T | NO T-T |
| | | | | MISMATCHES | MISMATCHES | MISMATCH | MISMATCH | BASE | MISMATCHES |
| | | | | ON BOTH | ARE | ON ONE | ON ONE | PAIRING ON | ARE |
| | | | | SIDES OF D | PRESENT IN | SIDE OF D | SIDE OF D | BOTH SIDES | PRESENT IN |
| | | | | | THE DUPLEX | | | OF D | THE DUPLEX |

### Example 3

### High-Throughput Screening of Non-Canonical Base Pairs

Concept: Using FIT-Probes as a High-Throughput Screen for Novel Metallo-Base Pairs

In this Example, 8 DNA sequences were synthesized, 4 with a single base replaced with quinoline blue and 4 complementary sequences. These sequences were designed such that combination of the dye-labeled sequences with the complementary sequences result in DNA duplexes containing a single base pair mismatch next to the dye. The sequences used are as follows (D denotes the dye):

| SEQ ID NO: | SEQUENCE (5′-3′) |
|---|---|
| 26 | ATA TTC TGA AT D ACA TCC TGC AT |
| 27 | ATA TTC TGA AA D ACA TCC TGCAT |
| 28 | ATA TTC TGA AG D ACA TCC TGCAT |
| 29 | ATA TTC TGA AC D ACA TCC TGCAT |
| 30 | ATG CAG GAT GT A ATT CAG AAT AT |
| 31 | ATG CAG GAT GT A TTT CAG AAT AT |
| 32 | ATG CAG GAT GT A GTT CAG AAT AT |
| 33 | ATG CAG GAT GT A CTT CAG AAT AT |

DNA duplexes were formed by mixing 0.5 uM of the single-stranded DNA in a 0.1 M NaNO₃ solution with its pH titrated to either 7 or 9. Metal ion solutions (HgCl₂, AgNO₃, CuNO₃, ZnNO₃, HAuCl₄, and MnCl₂ in water) were added to the DNA duplexes at 10- or 100-fold molar equivalents relative to the DNA. The fluorescence of the solutions was measured using a plate reader (excitation: 560 nm/emission: 610 nm) before (I₀) and after (I_{f}) the addition of the metal.

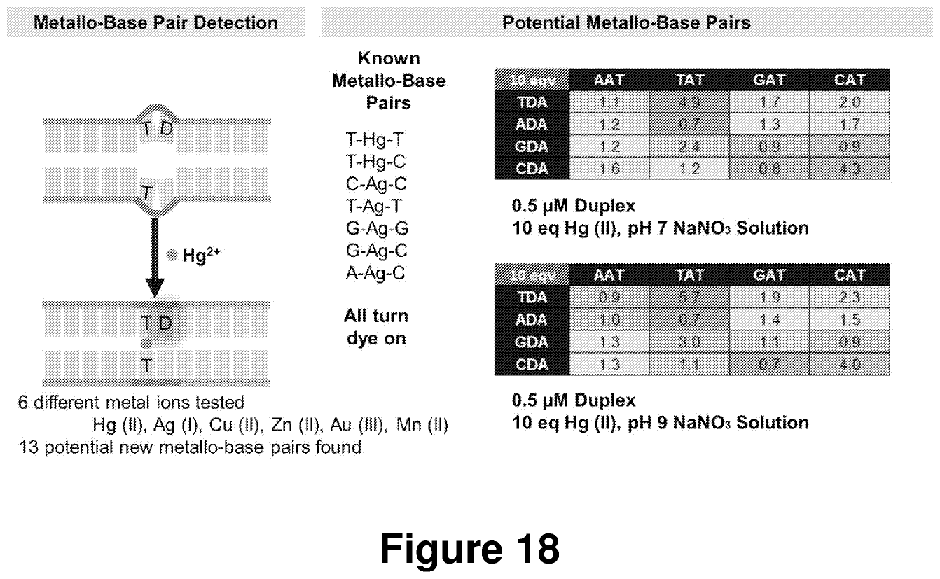

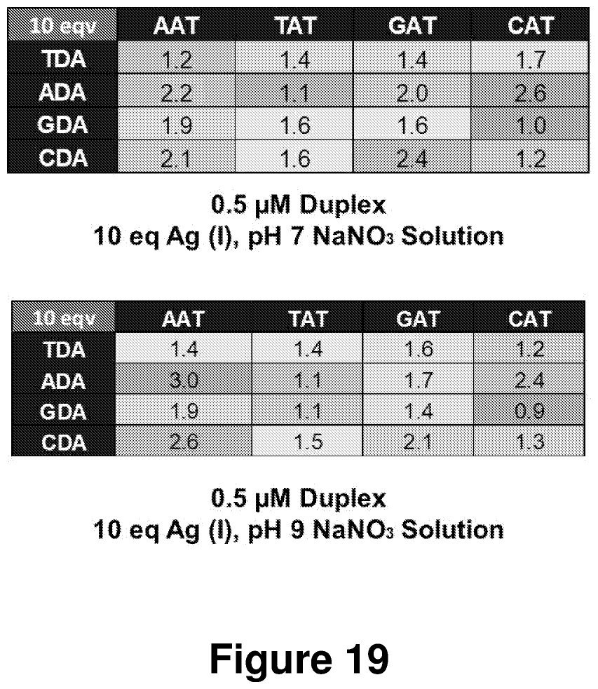

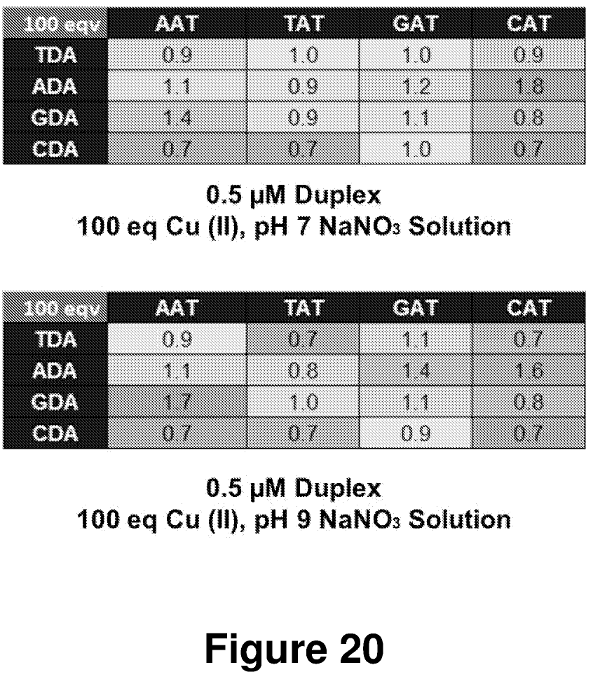

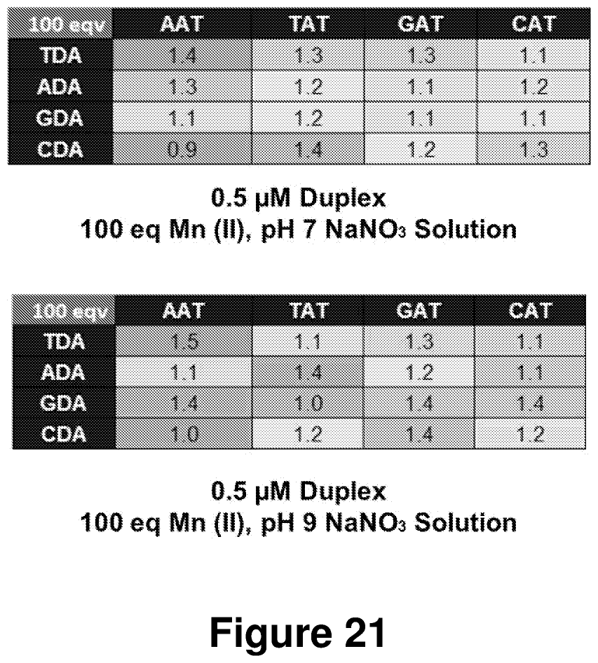

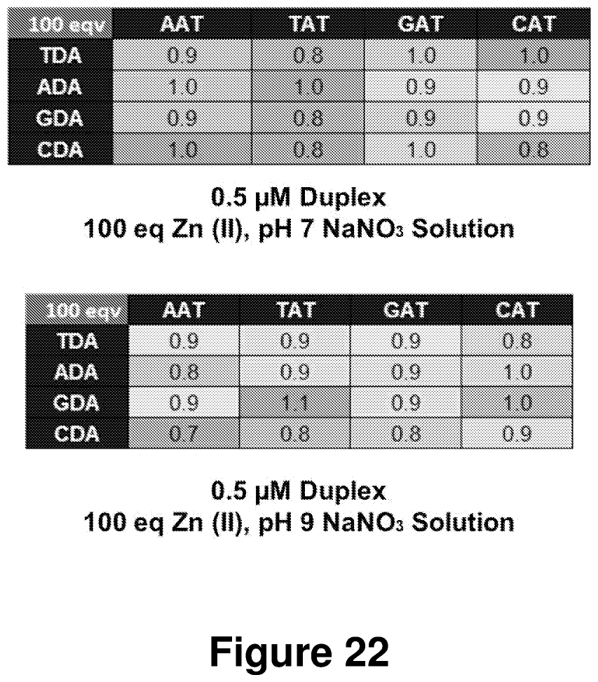

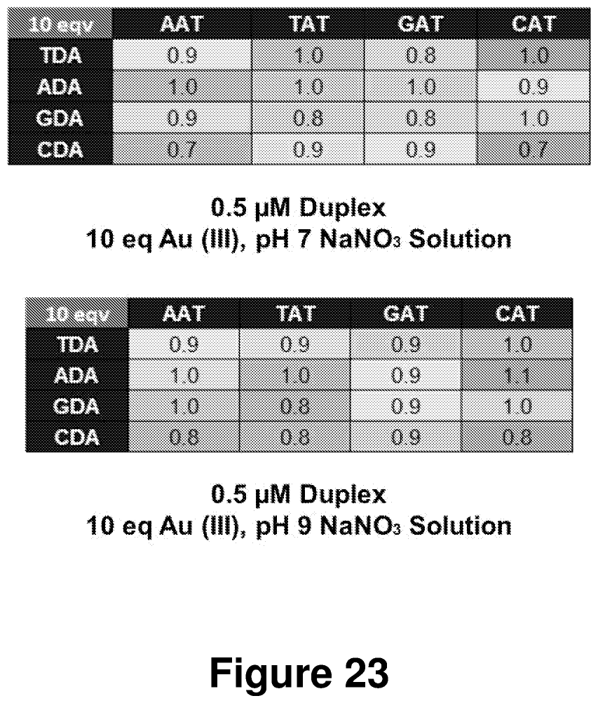

The fluorescence enhancement was calculated as I/l. A fluorescence enhancement was observed in several cases suggesting 13 potentially new metal-mediated base pairs. Results of the experiments are shown in FIGS. 18-23.

### CITATIONS REFERENCED IN EXAMPLES SECTION

(45) Wang, Z.; Heon Lee, J.; Lu, Y. Highly Sensitive “Turn-on” Fluorescent Sensor for Hg²⁺ in Aqueous Solution Based on Structure-Switching DNA. Chem. Commun. 2008, 7, 6005-6007.
(46) Heyduk, E.; Heyduk, T. Nucleic Acid-Based Fluorescence Sensors for Detecting Proteins. Anal. Chem. 2005, 77, 1147-1156.
(47) Chen, A.; Yan, M.; Yang, S. Split Aptamers and Their Applications in Sandwich Aptasensors. Trends Anal. Chem. 2016, 80, 581-593.
(48) Lartia, R.; Asseline, U. New Cyanine-Oligonucleotide Conjugates: Relationships between Chemical Structures and Properties. Chem.: Eur. J. 2006, 12, 2270-2281.
(49) Bethge, L.; Jarikote, D. V.; Seitz, O. New Cyanine Dyes as Base Surrogates in PNA: Forced Intercalation Probes (FIT-Probes) for Homogeneous SNP Detection. Bioorg. Med. Chem. 2008, 16, 114-125.
(50) Hövelmann, F.; Gaspar, I.; Chamiolo, J.; Kasper, M.; Steffen, J.; Ephrussi, A.; Seitz, O. LNA-Enhanced DNA FIT-Probes for Multicolour RNA Imaging. Chem. Sci. 2016, 7, 128-135.
(51) Haug, A.; Sauerbier, W. Influence of pH and Oxygen on 315 mμ Inactivation and Thymine Dimerization in Mutants of Bacteriophage T4. Photochem. Photobiol. 1965, 4, 555-561.
(52) Xia, T.; Yuan, J.; Fang, X. Conformational Dynamics of an ATP-Binding DNA Aptamer: A Single-Molecule Study. J. Phys. Chem. B 2013, 117, 14994-15003.
(53) US EPA, O. Drinking Water Contaminants—Standards and Regulations. Drink. Water Contam. 2013.

What is claimed is:

1. A method of detecting the presence of a target analyte comprising the step of contacting the target analyte with an aptamer comprising a detectable marker situated at an internal location within the aptamer, wherein the contacting results in binding of the target analyte to the aptamer,
wherein target analyte binding to the aptamer results in restriction of internal rotation of the marker, resulting in a detectable change in the marker.

2. The method of claim 1, wherein target analyte binding to the aptamer results in forced intercalation (FIT) of the marker between oligonucleotide base pairs of the aptamer.

3. A method of detecting the presence of a target analyte comprising the step of contacting the target analyte with
(a) an aptamer or portion thereof comprising (i) nucleotide sequence X, (ii) nucleotide sequence Y which binds to the target analyte, either alone or in combination with nucleotide sequence Y′ and (iii) a detectable marker situated at an internal location within the aptamer, and
(b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y,
wherein the contacting results in hybridization of nucleotide sequence X with nucleotide sequence X′ and binding of the target analyte with nucleotide sequence Y and nucleotide sequence Y′, wherein
the binding of nucleotide sequence X with nucleotide sequence X′ and the target analyte with nucleotide sequence Y and nucleotide sequence Y′ result in restriction of internal rotation of the marker, resulting in a detectable change in the marker.

4. The method of claim 3, wherein nucleotide sequence Y and nucleotide sequence Y′ bind to different binding sites of the target analyte.

5. The method of claim 3, wherein nucleotide sequence Y and nucleotide sequence Y′ together bind to the same binding site of the target analyte.

6. The method of any one of claims 3-5, wherein the binding of nucleotide sequence X with nucleotide sequence X′ and the target analyte with nucleotide sequence Y and nucleotide sequence Y′ result in forced intercalation (FIT) of the marker between oligonucleotide base pairs of the aptamer and the additional aptamer.

7. The method of any one of claims 1-6, wherein the detectable marker is a marker with internal rotation-dependent fluorescence.

8. The method of claim 7, wherein the detectable marker is a viscosity-sensitive marker.

9. The method of any one of claims 1-8, wherein the detectable marker is thiazole orange (TO), quinoline blue, quinoline violet, thiazole red, a derivative thereof, or a cyanine derivative.

10. The method of any one of claims 1-9, wherein the change in the detectable marker is proportional to concentration of the target analyte.

11. The method of any one of claims 1-10, wherein the target analyte is a protein, an ion, a small molecule, a lipid, a carbohydrate, an oligosaccharide, a cell, or a combination thereof.

12. The method of claim 11, wherein the ion is a metal ion.

13. The method of claim 12, wherein the metal ion is a mercury ion, a copper ion, a silver ion, zinc ion, gold ion, manganese ion, or a combination thereof.

14. The method of claim 11, wherein the ion is a hydrogen ion.

15. The method of claim 14, wherein the change in the detectable marker is indicative of a pH change.

16. The method of any one of claims 1-15, wherein the aptamer is a DNA aptamer, an RNA aptamer, or a modified form thereof.

17. The method of any one of claims 3-16, wherein the additional aptamer is a DNA aptamer, an RNA aptamer, or a modified form thereof.

18. The method of any one of claims 1-17, wherein the aptamer is about 5 to about 1000 nucleotides in length.

19. The method of any one of claims 1-18, wherein the aptamer is about 10 to about 100 nucleotides in length.

20. The method of any one of claims 2-19, wherein the additional aptamer is about 5 to about 1000 nucleotides in length.

21. The method of any one of claims 2-20, wherein the additional aptamer is about 10 to about 100 nucleotides in length.

22. The method of any one of claims 1-21, wherein the aptamer comprises a spacer.

23. The method of any one of claims 2-22, wherein the additional aptamer comprises a spacer.

24. The method of any one of claims 1-23, wherein the detectable marker is situated at a position that is x nucleotides from a terminus of the aptamer, wherein x is an integer that is 1, n/2, or any integer between 1 and n/2, wherein n is (i) the length of the aptamer and (ii) an even number.

25. The method of any one of claims 1-23, wherein the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number.

26. The method of any one of claims 1-25, wherein the detectable marker is situated at a position that is 1, 2, 3, 4, 5, 6, 7, 8, 9, or 10 nucleotides from a terminus of the aptamer.

27. A method of identifying a non-canonical base pair comprising the step of contacting an ion with
(a) a first oligonucleotide comprising a detectable marker situated at an internal location within the first oligonucleotide and
(b) a second oligonucleotide,
wherein the first oligonucleotide and the second oligonucleotide are sufficiently complementary to hybridize to each other and form a duplex, but are not complementary at a position in the duplex immediately adjacent to the detectable marker when the first oligonucleotide is hybridized to the second oligonucleotide; and
wherein binding of the ion to the duplex results in restriction in the internal rotation of the marker, resulting in a detectable change in the marker and thereby identifying the non-canonical base pair.

28. The method of claim 27, wherein binding of the ion to the duplex results in forced intercalation (FIT) of the marker in the duplex.

29. The method of claim 27 or claim 28, wherein the detectable marker is a marker with internal rotation-dependent fluorescence.

30. The method of claim 29, wherein the detectable marker with internal rotation-dependent fluorescence is a viscosity-sensitive marker.

31. The method of claim 29 or 30, wherein the detectable marker is thiazole orange (TO), quinoline blue, quinoline violet, thiazole red, a derivative thereof, or a cyanine derivative.

32. The method of any one of claims 27-31, wherein the ion is a cation.

33. The method of claim 32, wherein the cation is a metal ion.

34. The method of claim 33, wherein the metal ion is a mercury ion, a copper ion, a silver ion, zinc ion, gold ion, manganese ion, or a combination thereof.

35. The method of any one of claims 27-34, wherein the ion is an anion.

36. The method of any one of claims 27-35, wherein the first oligonucleotide is DNA, RNA, or a modified form thereof.

37. The method of any one of claims 27-36, wherein the second oligonucleotide is DNA, RNA, or a modified form thereof.

38. The method of any one of claims 27-37, wherein the first oligonucleotide is about 5 to about 1000 nucleotides in length.

39. The method of any one of claims 27-38, wherein the first oligonucleotide is about 10 to about 100 nucleotides in length.

40. The method of any one of claims 27-39, wherein the second oligonucleotide is about 5 to about 1000 nucleotides in length.

41. The method of any one of claims 27-40, wherein the second oligonucleotide is about 10 to about 100 nucleotides in length.

42. The method of any one of claims 27-41, wherein the first oligonucleotide comprises a spacer.

43. The method of any one of claims 27-42, wherein the second oligonucleotide comprises a spacer.

44. The method of any one of claims 27-43, wherein the detectable marker is situated at a position that is x nucleotides from a terminus of the first oligonucleotide, wherein x is an integer that is 1, n/2, or any integer between 1 and n/2, wherein n is (i) the length of the first oligonucleotide and (ii) an even number.

45. The method of any one of claims 27-43, wherein the detectable marker is situated at a position that is x nucleotides from a terminus of the first oligonucleotide, wherein x is an integer that is 1, (n+1)/2, or any integer between 1 and (n+1)/2, wherein n is (i) the length of the first oligonucleotide and (ii) an odd number.

46. The method of any one of claims 27-45, wherein the detectable marker is situated at a position that is 1, 2, 3, 4, 5, 6, 7, 8, 9, or 10 nucleotides from a terminus of the first oligonucleotide.

47. The method of any one of claims 27-46, wherein the first oligonucleotide and the second oligonucleotide are sufficiently complementary to hybridize to each other and form a duplex, but are not complementary at a single position in the duplex immediately adjacent to the detectable marker.

48. A method of identifying a non-canonical base pair comprising the step of contacting an ion with an aptamer comprising a detectable marker situated at an internal location within the aptamer,
wherein the aptamer is able to form an intramolecular duplex, and the duplex comprises a nucleotide mismatch at a position immediately adjacent to the detectable marker; and
wherein binding of the ion to the duplex results in restriction in the internal rotation of the marker, resulting in a detectable change in the marker and thereby identifying the non-canonical base pair.

49. The method of claim 48, wherein binding of the ion to the duplex results in forced intercalation (FIT) of the marker in the duplex.

50. The method of claim 48 or claim 49, wherein the detectable marker is a marker with internal rotation-dependent fluorescence.

51. The method of claim 50, wherein the detectable marker with internal rotation-dependent fluorescence is a viscosity-sensitive marker.

52. The method of claim 50 or 51, wherein the detectable marker is thiazole orange (TO), quinoline blue, quinoline violet, thiazole red, a derivative thereof, or a cyanine derivative.

53. The method of any one of claims 48-52, wherein the ion is a cation.

54. The method of claim 53, wherein the cation is a metal ion.

55. The method of claim 54, wherein the metal ion is a mercury ion, a copper ion, a silver ion, zinc ion, gold ion, manganese ion, or a combination thereof.

56. The method of any one of claims 48-55, wherein the ion is an anion.

57. The method of any one of claims 48-56, wherein the aptamer is a DNA aptamer, an RNA aptamer, or a modified form thereof.

58. The method of any one of claims 48-57, wherein the aptamer is about 5 to about 1000 nucleotides in length.

59. The method of any one of claims 48-58, wherein the aptamer is about 10 to about 100 nucleotides in length.

60. The method of any one of claims 48-59, wherein the aptamer comprises a spacer.

61. The method of any one of claims 48-60, wherein the detectable marker is situated at a position that is x nucleotides from a terminus of the aptamer, wherein x is an integer that is 1, n/2, or any integer between 1 and n/2, wherein n is (i) the length of the aptamer and (ii) an even number.

62. The method of any one of claims 48-60, wherein the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number.

63. The method of any one of claims 48-62, wherein the detectable marker is situated at a position that is 1, 2, 3, 4, 5, 6, 7, 8, 9, or 10 nucleotides from a terminus of the aptamer.

64. The method of any one of claims 48-63, wherein the duplex consists of a nucleotide mismatch at a position immediately adjacent to the detectable marker.


SEQUENCE LISTING (33 sequences)

SEQ ID NO: 1 - DNA, 32 bp, Artificial Sequence
  Synthetic Polynucleotide
  misc_feature (29)..(29): Dye
tcatgtttgtttgttggccccccttcttttta

SEQ ID NO: 2 - DNA, 13 bp, Artificial Sequence
  Synthetic Polynucleotide
caaacaaacatga

SEQ ID NO: 3 - DNA, 13 bp, Artificial Sequence
  Synthetic Polynucleotide
  misc_feature (3)..(3): Dye
ttttttttttttt

SEQ ID NO: 4 - DNA, 14 bp, Artificial Sequence
  Synthetic Polynucleotide
  misc_feature (14)..(14): Fluorescein
tttttttttttttt

SEQ ID NO: 5 - DNA, 14 bp, Artificial Sequence
  Synthetic Polynucleotide
  misc_feature (1)..(1): Tamra dT
tttttttttttttt

SEQ ID NO: 6 - DNA, 14 bp, Artificial Sequence
  Synthetic Polynucleotide
  misc_feature (1)..(1): Tamra dT
  misc_feature (14)..(44): Fluorescein
tttttttttttttt

SEQ ID NO: 7 - DNA, 17 bp, Artificial Sequence
  Synthetic Polynucleotide
  misc_feature (3)..(3): Dye
ccccccaaaaccctccc

SEQ ID NO: 8 - DNA, 24 bp, Artificial Sequence
  Synthetic Polynucleotide
  misc_feature (8)..(8): Dye
cccctaaccctaacccctaacccc

SEQ ID NO: 9 - DNA, 21 bp, Artificial Sequence
  Synthetic Polynucleotide
  misc_feature (15)..(15): (Spacer 18)5
  misc_feature (18)..(18): Dye
ggttggtgtggttggcgctct

SEQ ID NO: 10 - DNA, 36 bp, Artificial Sequence
  Synthetic Polynucleotide
  misc_feature (7)..(7): (Spacer 18)5
agatgcgagtccgtggtagggcaggttggggtgact

SEQ ID NO: 11 - DNA, 22 bp, Artificial Sequence
  Synthetic Polynucleotide
  misc_feature (15)..(15): (Spacer 18)5
  misc_feature (22)..(22): Fluorescein
ggttggtgtggttggcgcatct

SEQ ID NO: 12 - DNA, 36 bp, Artificial Sequence; identical to SEQ ID NO: 10
  Synthetic Polynucleotide
  misc_feature (1)..(1): Tamra dT
  misc_feature (7)..(7): (Spacer 18)5

SEQ ID NO: 13 - DNA, 22 bp, Artificial Sequence
  Synthetic Polynucleotide
  misc_feature (15)..(15): (Spacer 18)5
ggttggtgtggttggcgcatct

SEQ ID NO: 14 - DNA, 37 bp, Artificial Sequence
  Synthetic Polynucleotide
  misc_feature (8)..(8): (Spacer 18)5
tagatgcgagtccgtggtagggcaggttggggtgact

SEQ ID NO: 15 - DNA, 19 bp, Artificial Sequence
  Synthetic Polynucleotide
  misc_feature (13)..(13): (Spacer 18)5
  misc_feature (16)..(16): Dye
tggttggttttttcgctct

SEQ ID NO: 16 - DNA, 21 bp, Artificial Sequence
  Synthetic Polynucleotide
  misc_feature (7)..(7): (Spacer 18)5
agatgcgaaaaaaggttggtg

SEQ ID NO: 17 - DNA, 20 bp, Artificial Sequence
  Synthetic Polynucleotide
  misc_feature (11)..(11): Dye
atattctgaattcatcctgc

SEQ ID NO: 18 - DNA, 20 bp, Artificial Sequence
  Synthetic Polynucleotide
  misc_feature (11)..(11): Dye
atattctgaatacatcctgc

SEQ ID NO: 19 - DNA, 20 bp, Artificial Sequence
  Synthetic Polynucleotide
  misc_feature (11)..(11): Dye
atattctgaaaacatcctgc

SEQ ID NO: 20 - DNA, 21 bp, Artificial Sequence
  Synthetic Polynucleotide
tataagactttgtgtaggacg

SEQ ID NO: 21 - DNA, 21 bp, Artificial Sequence
  Synthetic Polynucleotide
tataagactttgagtaggacg

SEQ ID NO: 22 - DNA, 21 bp, Artificial Sequence
  Synthetic Polynucleotide
tataagacttagagtaggacg

SEQ ID NO: 23 - DNA, 33 bp, Artificial Sequence
  Synthetic Polynucleotide
tcatgtttgtttgttggccccccttctttctta

SEQ ID NO: 24 - DNA, 13 bp, Artificial Sequence
  Synthetic Polynucleotide
  misc_feature (3)..(3): Dye
ccccccccctccc

SEQ ID NO: 25 - DNA, 15 bp, Artificial Sequence
  Synthetic Polynucleotide
  misc_feature (5)..(5): Dye
ccccccccccccccc

SEQ ID NO: 26 - DNA, 22 bp, Artificial Sequence
  Synthetic Polynucleotide
  misc_feature (11)..(11): Dye
atattctgaatacatcctgcat

SEQ ID NO: 27 - DNA, 23 bp, Artificial Sequence
  Synthetic Polynucleotide
  misc_feature (11)..(11): Dye
atattctgaaadacatcctgcat

SEQ ID NO: 28 - DNA, 23 bp, Artificial Sequence
  Synthetic Polynucleotide
  misc_feature (11)..(11): Dye
atattctgaagdacatcctgcat

SEQ ID NO: 29 - DNA, 22 bp, Artificial Sequence
  Synthetic Polynucleotide
  misc_feature (11)..(11): Dye
atattctgaacacatcctgcat

SEQ ID NO: 30 - DNA, 23 bp, Artificial Sequence
  Synthetic Polynucleotide
atgcaggatgtaattcagaatat

SEQ ID NO: 31 - DNA, 23 bp, Artificial Sequence
  Synthetic Polynucleotide
atgcaggatgtatttcagaatat

SEQ ID NO: 32 - DNA, 23 bp, Artificial Sequence
  Synthetic Polynucleotide
atgcaggatgtagttcagaatat

SEQ ID NO: 33 - DNA, 23 bp, Artificial Sequence
  Synthetic Polynucleotide
atgcaggatgtacttcagaatat